SEQUENCE LISTING (16 sequences)

SEQ ID NO: 1 - DNA, 28 bp, Homo sapiens
ccgcgccacttggcctgcctccgtcccg

SEQ ID NO: 2 - DNA, 30 bp, Homo sapiens
cgggatccatgaaggggcagcagaaaacag

SEQ ID NO: 3 - DNA, 34 bp, Homo sapiens
gctctagattagttgctgtcattcttgatgacga

SEQ ID NO: 4 - DNA, 32 bp, Homo sapiens
ccggaattccatgccatggacatgctggaccc

SEQ ID NO: 5 - DNA, 32 bp, Homo sapiens
gctctagacatgtgtccctctctgtgctaagg

SEQ ID NO: 6 - DNA, 28 bp, Homo sapiens
ccgcgccacttggcctgcctccgtcccg

SEQ ID NO: 7 - DNA, 28 bp, Homo sapiens
ccgcgccacttcgcctgcctccgtcccg

SEQ ID NO: 8 - DNA, 23 bp, Homo sapiens
cacccggtcacgtggcctacacc

SEQ ID NO: 9 - DNA, 23 bp, Homo sapiens
cacccggtcaattggcctacacc

SEQ ID NO: 10 - DNA, 28 bp, Homo sapiens
gtcctgccaccgcgcgtcttggcctgcc

SEQ ID NO: 11 - DNA, 19 bp, Homo sapiens
cgagcaggaagaggcggag

SEQ ID NO: 12 - DNA, 20 bp, Homo sapiens
tccgagccggccacaggcat

SEQ ID NO: 13 - DNA, 21 bp, Homo sapiens
gcctgaagtctctgattaagt

SEQ ID NO: 14 - DNA, 19 bp, Homo sapiens
acacctgcgtcgaagatgt

SEQ ID NO: 15 - DNA, 30 bp, Homo sapiens
cccctggccaaggtcatccatgacaacttt

SEQ ID NO: 16 - DNA, 30 bp, Homo sapiens
ggccatgaggtccaccaccctgttgctgta

(19) United States
(12) Patent Application Publication
(10) Pub. No.: US 2006/0051764 A1
(43) Pub. Date: Mar. 9, 2006

(54) Thymidylate synthase polymorphisms for use in screening for cancer susceptibility

(76) Inventors: Michael Mandola, Lakewood, NJ (US); Jan Stoehlmacher, Hamburg (DE); Heinz-Josef Lenz, Altadena, CA (US); Robert Ladner, Haddonfield, NJ (US)

Correspondence Address: LICATA & TYRRELL P.C., 66 EAST MAIN STREET, MARLTON, NJ, 08053, US

(21) Appl. No.: 10/532,201

(22) PCT Filed: Oct. 21, 2003
(86) PCT No.: PCT/US03/33441

§ 371 Date: Jun. 27, 2005

Related U.S. Application Data
(60) Provisional application No. 60/420,164, filed on Oct. 21, 2002.

(51) Int. Cl. C12Q 1/68; C07H 21/04

(52) U.S. Cl. 435/6; 536/24.3

(57) ABSTRACT

The present invention discloses a novel single nucleotide polymorphism (SNP) in the isolated 5′ tandem repeats of the thymidylate synthase (TS) gene and methods for its use. The novel SNP, located in the 12th nucleotide of a 28 bp third tandem repeat (3R) of the TS gene, substitutes a C for a G, and is the variant form of the repeat. Subjects with the wild-type form of 3R have greater transcription of the TS gene than subjects with the variant form. The invention also reveals that a six base pair deletion in the 3′ region of TS (−6 bp/1494) indicates mRNA instability and thus reduced production of TS. In diseased tissue, such as cancer, reduced production of TS is beneficial because it prevents the cancerous cells from growing and spreading. Analysis of either polymorphism or both together allows for prediction of a subject's response to chemotherapeutic and anti-cardiovascular disease treatments because both diseases are related to TS levels in a subject.

### CROSS-REFERENCE TO RELATED APPLICATIONS

The present application is a continuation-in-part of United States Provisional Application No. 60/420,164, entitled “A Novel Single Nucleotide Polymorphism in the Tandem Repeats of the Thymidylate Synthase Gene Alters USF-1 Binding and Transcriptional Activation,” filed Oct. 21, 2002, which is incorporated by reference in its entirety herein.

### TECHNICAL FIELD

The present invention relates to the field of medical genetics and disease susceptibility screening. Specifically, the present invention relates to the identification, prognostic use and therapeutic use of a single nucleotide polymorphism in the 5 region of thymidylate synthase (TS) gene. The polymorphism indicates the transcriptional activity of the TS gene, and relatedly, the risk of cancer and cardiovascular disease. The invention also relates to the prognostic and therapeutic use of and screening methods for a six base pair polymorphism found in the 3′ untranslated region of TS.

### BACKGROUND OF THE INVENTION

Thymidylate synthase (TS) is an important enzyme in the nucleotide biosynthetic pathway that converts dUMP to dTMP via reductive methylation. The TS reaction is the only source of de novo thymidylate in the cell and is thus essential for DNA replication (Friedkin, et al., 1957; Heidelberger et al., 1957; Santi et al., 1984). The critical role of TS in nucleotide metabolism has made it a common target for a variety of chemotherapeutic agents including 5-fluorouracil (5-FU), raltitrexed (Tomudex), capecitabine (Xeloda), and pemetrexed (Alimta) (Danenberg, 1977; Papamichael, 1999). Inhibition of TS by these agents leads to cytotoxicity induced by dTTP pool depletion leading to thymineless death (Houghton, 1999), and in some instances uracil misincorporation into DNA (Aherne, 1999; Ladner, 2001), which causes irreparable strand breaks through the action of uracil-DNA-glycosylase. Limited efficacy of TS inhibitors in the treatment of human cancers has been a common phenomenon. Resistance to fluoropyrimidines arises through a variety of mechanisms, including increases in TS transcription (Shibata et al. 1998) and translation (Kaneda, et al., 1987; Keyomarsi et al., 1993).

With respect to the relationship between TS, cardiovascular disease (CVD), and other defects, TS and an enzyme called methylenetetrahydrofolate reductase (MTHFR) compete for limited supplies of folate required for the remethylation of homocysteine (Trinh, 2002). Low plasma folate and high homocysteine levels have been independently and collectively correlated with an increased risk of CVD. Specifically, elevated plasma homocysteine is a known risk factor for occlusive vascular disease, venous thrombosis, neural tube defects and pregnancy complications.

A polymorphism within the 5′-untranslated region of the TS gene, consisting of tandem repeats of 28 base pairs, has been implicated in modulating TS mRNA expression (Kandea, et al., 1987; Horie et al., 1995) and TS mRNA translational efficiency (Kawakami, et al., 2001). Although there have been reports of 4, 5 and 9 repeats within certain African and Asian populations (Marsh et al., 1999; Marsh et al., 2000; Luo et al., 2002), the majority of individual human TS alleles harbor either a double repeat (2R) or a triple repeat (3R) for this polymorphism creating genotypes of 2R/2R, 2R/3R and 3R/3R. Individuals that are homozygous for the 3R were found to have elevated intratumoral TS mRNA (Pullarkat et al., 2001) and protein levels compared to 2R homozygotes (Kawakami et al., 1999).

In addition, the 5′ tandem repeat polymorphism of the TS gene has been identified as a predictor of clinical outcome to 5-FU based chemotherapy in both adjuvant and metastatic settings (Pullarkat et al., 2001; Villafranca et al., 2001; Marsh et al., 2001; Iacopetta et al., 2001) as well being associated with predicting risk and outcome of acute lymphoblastic leukemia (Krajinovic et al., 2002; Skibola et al., 2002). The tandem repeats have also been shown to predict plasma folate and homocysteine levels (Trinh et al., 2002) and the risk of colorectal adenomas (Ulrich et al., Cancer Res., 2002). Although screening for the 5′ tandem repeats alone has shown great promise, the need for more accurate and comprehensive screens is warranted. In particular, the identification of novel functional polymorphisms that can be added to already useful tests may help enhance the predictive value of the tests. Testing for several polymorphisms in conjunction with each other may further increase the predictive value of determining an individual's risk for cancer or CVD and also his response to known treatments for the disease.

USF-1 and USF-2 (upstream stimulatory factors) belong to a family of transcriptional regulatory factors bearing helix-loop-helix domains, similar to cMyc, and are found together to a large extent as heterodimers in the cell (Sirito, et al., 1992; Viollet et al., 1996). The E-box is a consensus element for the helix-loop-helix USF transcriptional activator family of proteins (Singh et al., 1994; Kiermaier et al., 1999; Luo et al., 1996; Ferre-D'Amare et al., 1994). The DNA binding activity of USF-1 to E-box (CANNTG) consensus sequences is regulated through phosphorylation by cdc2/p34 (Cheung et al., 1999) and the stress-responsive p38 kinase (Galibert et al., 2001). Phosphorylation of USF-1 by these kinases has been shown to activate USF-1 transcriptional activity under normal and stressful conditions, respectively.

Through its DNA binding activity, USF-1 has been shown to transactivate a variety of genes including p53 (Reisman et al., 1993) and the Adenomatous Polyposis Coli (APC) protein (Jaiswal, et al., 2001). Although both USF-1 and USF-2 were thought to be ubiquitously expressed factors, recent evidence suggests that USF-1 and USF-2 are differentially regulated in some cancer cells (Ismail et al., 1999). The differential regulation may have an effect on the ability of USF-1/USF-2 complexes to form and function properly.

Another polymorphism within the TS gene, consisting of a 6 bp deletion of the sequence TTAAAG at nucleotide 1494 of the TS mRNA (“−6 bp/1494”), has been recently discovered through searching the public Expressed Sequence Tag (EST) database (Ulrich, 2000). This common polymorphism is also transcribed into the 3′UTR of the primary TS transcript. Little is currently known about the 3′UTR of TS. 3′UTRs function as post-transcriptional regulators mainly through control of mRNA stability and/or translational efficiency, and are thought to play an important role in the overall fate of mRNAs (Grzybowska, 2001). Traditionally, it was thought that the function of 3′UTRs was governed primarily through mRNA secondary structural elements, such as stem loop structures.

Although this remains true, recent evidence has shown a growing number of cis-binding sequence elements within 3′UTRs that interact with RNA-binding regulatory proteins in a sequence specific manner. In fact, the regulation of some well characterized mRNAs have been shown to be dependent, in part, on cis-binding sequences within the 3′UTR. For example, the 3′UTRs of COX-2 and p21^{WAF1} mRNAs have been shown to be essential for the proper post-transcriptional regulation of these transcripts (Cok, 2001; Giles, 2003). Further, polymorphisms in the 3′UTRs of other mRNAs have been shown to have a functional effect on overall gene expression. A polymorphism in the 3′UTR of the dihydrofolate reductase mRNA, which encodes a critical enzyme that is involved in folate metabolism, plays a functional role in governing the post-transcriptional regulation of the mRNA and in the overall regulation of gene expression (Goto, 2001).

Until this point, the molecular mechanism by which the 5′ tandem-repeat polymorphism enhances transcription has not yet been elucidated. Further, differences in the nucleotide sequences of the repeats have not been considered as playing a functional role in transcription and post-transcriptional events. It would be a significant improvement in the art to identify the regulatory factor(s) responsible for binding within the polymorphic region and enhancing TS mRNA expression. This improvement would allow an understanding of why 3R repeats show increased TS transcription as compared to 2R. It would then permit diagnostic and therapeutic use of the functional difference to identify and treat patients at risk for diseases, such as cancer and CVD, related to the TS pathway, which would result in significantly improved and targeted treatments.

Additionally, the 3′UTR of TS mRNA has not been studied up to this point as playing a functional role in post-transcriptional regulation. Further, the molecular mechanism(s) by which the −6 bp/1494 deletion polymorphism may affect the regulation of TS mRNA has yet to be elucidated. Characterizing the regions within the TS 3′UTR that may be responsible for the post-transcriptional regulation of TS mRNA, and to identifying the mechanism(s) by which the deletion polymorphism affected TS mRNA regulation would be a significant discovery in this area. Revealing the effect that the 6 base pair polymorphism has in the 3′ UTR of TS RNA provides an additional screen for predicting an individual's TS level. It also improves the chances of success of targeted clinical therapies, including cancer therapies directed to blocking TS creation or function.

### SUMMARY OF THE INVENTION

A first aspect of the invention identifies a novel single-nucleotide polymorphism (SNP) within the third tandem repeat that determines the binding and transactivating ability of USF complexes and occurs at a high frequency in the tested population. The clinical data shows that the screening for the G to C SNP in combination with the tandem repeat polymorphism (3RV) significantly increases the value of the tandem repeats in predicting response and survival to cancer treatment, particularly 5-FU/LV. Individuals with two regular 3R copies have the worst response. 3RV copies increase the response to treatment for cancer and/or CVD.

In an additional aspect of the invention, USF-1 and USF-2 are identified as factors that bind within the tandem repeat polymorphism of the TS 5′ regulatory region.

A third aspect of the invention shows that USF-1 enhances transcription of 2R, 3R and 3RV TS reporter gene constructs in a luciferase assay system and that the impact of a 2R or 3R genotype on TS transcriptional activation is ultimately related to the presence or absence of the USF binding sites.

Yet another aspect of the invention encompasses a diagnostic kit for screening for cancer and/or cardiovascular risk by examining the TS SNP polymorphism in conjunction with the 5′ tandem repeat polymorphism alone, the 3′ −6 bp/1494 polymorphism alone, or using both the TS polymorphisms in conjunction with each other. The screen uses the genetic material of an individual to examine which polymorphism, or combination of polymorphisms, exists in that individual. The diagnostic kit comprises one or more relevant diagnostic primers or probes and/or an allele-specific oligonucleotide primers of the invention. The kit may also comprise packaging, vials and tubes, instructions for use, buffer, polymerase, and/or other reaction components.

In a further aspect, the diagnostic methods of the invention are used to predict the chance of an individual developing cancer and/or CVD. Relatedly; screening for the polymorphisms, alone or in combination, and can predict the efficacy of therapeutic compounds in the treatment of cancer and cardiovascular-related diseases via use of high throughput screening (HTS). The HTS rapidly and efficiently screens multiple patients for cancer and/or cardiovascular risk. For example, if an individual has a lower rate of transcription of TS, that person likely has a lesser chance of developing tumors and a better chance of fighting/shrinking the tumors that currently exist.

A related aspect of the present invention is the pharmacogenetic use of the TS SNP and tandem repeats and/or −6 bp/1494 polymorphism to identify patients most suited to therapy with particular pharmaceutical agents and use of the TS SNP in pharmaceutical research to assist the drug selection process.

Another object of the invention is to provide a useful target for linkage analysis and disease association studies.

Yet another object is to develop a novel molecular diagnostic markers useful in the detection of CVD and cancer.

Another aspect of the invention comprises the use of gene alteration or replacement to induce the polymorphisms that produce the desired transcription with respect to the TS gene. For example, if reduced activity were desired, the TS gene would be manipulated to include those sequences that result in reduced transcription and/or activity.

Another aspect of the invention is blocking the production and/or activity of the TS enzyme in the target cells.

### BRIEF DESCRIPTION OF THE DRAWINGS

FIG. 1 is a sequence depiction of a tandem repeat polymorphism within the 5′-untranslated region of the human TS gene. The position of the E-box is indicated.

FIG. 2 is a gel showing that USF proteins bind to the E-box site within the tandem repeats of the human TS gene in HT29 nuclear extracts.

FIG. 3 is a picture of four gels displaying different aspects of USF-1 activity. FIG. 3A shows phosphorylation of recombinant USF-1 by cdc2/p34 in vitro. FIG. 3B shows that phosphorylated recombinant USF-1 binds to its consensus sequence by EMSA. FIG. 3C shows that phospho-USF-1 binds to the TS tandem repeats bearing an E-box site. Finally, FIG. 3D shows that USF-1 does not bind to the variant TS tandem repeats with a G→C base change at the 12^{th} nucleotide.

FIG. 4 is the result of a ChIP assay, demonstrating that USF-1 and USF-2 bind to the TS 5′ UTR in vivo.

FIG. 5A is a diagram of the structure of the TS luciferase reporter constructs. FIG. 5B is a bar chart showing the levels of activation of the TS gene promoter by USF-1.

FIG. 6A is the HaeIII restriction map of the TS tandem repeat fragments produced in the RFLP analysis. FIG. 6B is a gel showing the results of a restriction fragment length polymorphism (RFLP) analysis used for screening of the tandem repeats, as well as the G→C SNP.

FIG. 7 shows that the 3′UTR of TS contains no elements of transcript instability or translational silencing. FIG. 7A is the structure of the chimeric luciferase reporter constructs bearing proximal and distal end deletions of the TS 3′UTR. The TS 3′UTR sequences were inserted between the luciferase coding region and poly(A) signal. Transcription was controlled by the SV40 promoter in all constructs. The luciferase gene is indicated in the white bars and the TS 3′UTR regions are shown in black bars. The numbers indicate the region of TS 3′UTR that was inserted, and numbering begins just after the stop codon of TS.

FIG. 7B shows the activity and mRNA levels of the TS 3′UTR reporter constructs. Luciferase activity (black bars) was normalized to β-Galactosidase activity and is expressed as a percentage of the activity from the empty pGL3-control vector. Luciferase mRNA levels (white bars) were normalized to internal GAPDH mRNA levels and are expressed as a percentage of the mRNA levels of the empty pGL3-control vector. All results are the mean±S.E. for 3 independent experiments, each measured in duplicate. a=significantly different (p<0.005) from pGL3-control; b=significantly different (p<0.05) from pGL3-control.

FIG. 8 demonstrates that the −6 bp/1494 deletion polymorphism causes decreased luciferase activity and message levels compared to +6 bp/1494 constructs. FIG. 8A shows the structure of the chimeric luciferase reporter constructs bearing proximal end deletions of the TS 3′UTR that contain either the +6 bp/1494 insertion or the −6 bp/1494 deletion polymorphism. The deletion polymorphism lies at nucleotide 456 of the TS 3′UTR. The TS 3′UTR sequences were inserted between the luciferase coding region and poly(A) signal. Transcription was controlled by the SV40 promoter in all constructs. The luciferase gene is indicated in the white bars and the TS 3′UTR regions are shown in black bars (gaps indicate the −6 bp/1494 deletion polymorphism). The numbers indicate the region of TS 3′UTR that was inserted, and numbering begins just after the stop termination codon of TS. The brackets indicate the construct counterparts that should be compared and the + and − indicate that the constructs contain either the +6 bp/1494 or −6 bp/1494 polymorphism.

FIG. 8B shows the activity and mRNA levels of the TS 3′UTR reporter constructs. Luciferase activity (black bars) was normalized to β-Galactosidase activity and is expressed as a percentage of the activity from the empty pGL3-control vector. Luciferase mRNA levels (white bars) were normalized to internal GAPDH mRNA levels and are expressed as a percentage of the mRNA levels of the empty pGL3-control vector. The brackets indicate the construct counterparts that should be compared and the + and − indicate that the construct contains either the +6 bp/1494 or −6 bp/1494 polymorphism. All results are the mean±S.E. for 3 independent experiments, each measured in duplicate. a=significantly different (p<0.005) from pGL3-control; b=significantly different (p<0.05) from pGL3-control; c=significantly different (p<0.005) from respective +6 bp/1494 counterpart; d=significantly different (p<0.05) from respective +6 bp/1494 counterpart. Inset shows a representative RT-PCR of chimeric message electrophoresed on a 2% agarose gel. The internal GAPDH control (510 bp) PCR was run in the same reaction as the luciferase (97 bp) PCR.

FIG. 9 shows that the −6 bp/1494 deletion polymorphism causes decreased mRNA stability. 293 cells were transfected with reporter gene constructs and treated with actinomycin D (final concentration of 10 μg/ml) 24 hours post-transfection. Total RNA was extracted at various time points for 6 hours, reverse transcribed and assayed for luciferase and GAPDH levels by semi-quantitative PCR. Luciferase mRNA levels were normalized to GAPDH message and are expressed as a percentage of the mRNA present at the 0 h time point (100%). All experiments and time points are the results of three independent experiments performed in duplicate. Asterisks (*) indicate that the message levels of the −6 bp/1494 constructs were significantly different (p<0.05) from their +6 bp/1494 counterparts at the 2, 4 and 6 hour time points.

### DETAILED DESCRIPTION OF THE INVENTION

I. Overview

A first aspect of the invention is the discovery of the isolated nucleic acid comprising a thymidylate synthase single nucleotide polymorphism (TS SNP), and probes and primers therefor. “Isolated” means not naturally occurring. “Isolated nucleic acid” means a nucleic acid that is not immediately contiguous with the 5′ and 3′ flanking sequences with which it normally is immediately contiguous when present in the naturally occurring genome of the organism from which it is derived. “Isolated nucleic acid” may describe a nucleic acid that is incorporated into a vector, incorporated into the genome of a heterologous cell, or that exists as a separate molecule. The phrase may also describe a recombinant nucleic acid that forms part of a hybrid gene encoding additional polypeptide sequences that may be used to produce a fusion protein. Thus, the TS SNP isolated nucleic acid may take any of these forms.

Further, there is the potential to create and utilize probes to and primers for the TS 5′ SNP and/or the 3′ −6 bp/1494 polymorphism. A probe for the TS SNP could be created such that the probe would only bind to the variant form of the TS tandem repeats comprising the TS SNP. A probe for the 3′ −6 bp/1494 polymorphism could be created such that the probe would only bind to the wild type form (+6 bp/1494) or only bind to the variant form (−6 bp/1494) of the polymorphism. The probe may bind to any purified or nonpurified nucleic acid portion may be used if it contains the TS gene or more specifically, contains the polymorphic portions of the TS gene of interest. The nucleic acid may be single or double stranded DNA or RNA, including messenger RNA.

A probe is the term for a piece of DNA or RNA corresponding to the gene or sequence of interest. Here, the sequence of interest is the third tandem repeat in the 5′ region of the TS gene and/or the 3′ untranslated region of TS. The first probe has a sequence that is complementary to the sequence of the isolated nucleic acid of interest, and which selectively binds to the variant form of the tandem repeat but not to the wild-type form of the tandem repeat. The second probe has a sequence that is complementary to the sequence of the isolated nucleic acid of interest, and which selectively binds to the selected form of the 3′ UTR of the TS, but not to the alternate form. Preferably, the probe is labeled for easy detection. Labels, for example, biotin, digoxygenin, or fluorescein, and methods for their attachment to the probe are known in the art.

Another embodiment of the present invention are primers for the nucleic acid comprising the TS SNP and/or the 3′ UTR polymorphism, which like a probe, hybridizes to the sequence of interest. However, primers also allow for extension of the nucleic acid sequence with the addition of free nucleotides, polymerase, and other necessary reagents into the reaction mixture. Hybridization means selectively binding to a nucleotide sequence under stringent conditions. Here, the stringent conditions are those that permit the binding the variant 3R tandem repeat, but not the wild-type 3R tandem repeat, or vice versa. With respect to the 3′ UTR polymorphism, stringent conditions are those that permit the binding of one form of the polymorphism, but not the other.

Primers are used typically within an amplification procedure, such as PCR. For polymerase chain reaction (PCR) amplification of regions to TS gene containing a polymorphism, nucleoside triphosphates (dATP, dCTP, dGTP, and dTTP), a polymerizing agent and proper temperature, ionic strength and pH are required. Preferably, the primer is single-stranded and sufficiently long to allow synthesis via extension using the polymerizing agent. The oligonucleotide primer typically contains at least 8-40 nucleotides, but preferably 12-35 nucleotides. PCR allows for exponential amplification of a portion of nucleic acid.

Primers should be “substantially” complementary to the nucleic acid being amplified, meaning that the primers must be sufficiently complementary to hybridize with their respective strands and permit the amplification to occur. The primers may be prepared using conventional or automated phosphotriester and phosphodiester methods. Preferably, the primer extension is performed in the presence of A, C, G, and T/U nucleotide terminators, each of which is labeled with a different label that identifies the base contained in the terminator. A nucleotide terminator is a nucleotide or nucleoside that is covalently linkable to the extendible end of a primer, but is not capable of further extension. Preferably, the labels are fluorescent labels with four different emission wavelengths.

PCR proceeds with primers to denatured nucleic acid followed by extension with polymerase or another enzyme and then undergoes repeated cycles of denaturing, primer annealing, and extension. Specific conditions for the PCR may be found in the “Experimental” section or are known in the art. The final amplified regions of TS may be detected by Southern blots with or without using radioactive probes. A “region” is an area from several nucleotides upstream to several nucleotides downstream from the specific nucleotide mentioned and also includes the complementary nucleotides on the antisense strand of sample DNA. Nonradioactive probes include a fluorescent compound, a bioluminescent compound, a chemiluminescent compound, a metal chelator or an enzyme. The amplification products can also be separated using an agarose gel containing ethidium bromide.

Relatedly, the probe may be part of a nucleic acid array in which an oligonucleotide hybridizes to the sequence comprising the TS SNP and/or the 3′ UTR polymorphism. In this embodiment, an array of nucleic acid molecule targets is attached to a solid support. If the array is screening for the 5′ TS SNP, it comprises an oligonucleotide that will hybridize to a nucleic acid molecule consisting of CCGCGCCACTTGGCCTGCCTCCGTCCCG [SEQ ID NO:1], wherein at position 12, G is replaced by C, under conditions in which the oligonucleotide will not substantially hybridize to a nucleic acid molecule consisting of SEQ ID NO:1. If the array is screening for the 3′ UTR polymorphism, it comprises an oligonucleotide that will hybridize to a nucleic acid molecule having one of the forms of the polymorphism. For example, the array may comprise an oligonucleotide that will hybridize to a molecule having a +6 bp/1494 region, but not to a molecule having a −6 bp/1494 region. An array may also be designed where the converse is true: an oligonucleotide that will hybridize to a molecule having a −6 bp/1494 region, but not to a molecule having a +6 bp/1494 region. An array may have one or a plurality of target elements, including, but not limited to both the TS targets revealed herein.

A different aspect of the present invention focuses on the upstream regulatory factors (USF-1 and USF-2) that bind to the key regions of the tandem repeats, called E-boxes. The present invention contemplates manipulation of the binding of these USF elements, both through manipulation of the USF elements themselves and their binding regions. For example, since USF binding leads to TS transcription, which in turn, leads to increased risk of cancer and cardiovascular disease, the USF binding elements themselves could be altered so as not to bind with the efficacy or frequency of wild-type USF elements. This alteration could occur through mutation of the nucleic acid that codes for the USF, through blocking the protein assembly, or through alteration of the USF proteins function after assembly. Additionally, any alteration of the E-box of the tandem repeat sequences that prevents USF binding, specifically the G for C substitution at the 12^{th} nucleotide of third tandem repeat, is contemplated. Any alteration that prevents the binding of the USF factors is within the scope of the present invention.

The next aspects of the invention relate to methods of using the novel TS SNP in the 5′ region and/or the 3′ UTR polymorphism to discover disease susceptibility of an individual not having a disease and optimal disease treatment pathways including drug selection for an individual having a disease. Further, the methods contemplated comprise using the polymorphisms as molecular markers and for linkage analysis and using genetic manipulation to better an individual's chances of surviving a disease or of not contracting a disease at all. The diseases focused on in the present invention are cancer and cardiovascular disease, although the methods of the invention are applicable to any other disease in which thymidylate synthase is implicated.

To identify the TS polymorphisms, nucleic acid must first be extracted from the subject. Preferably, the subject is a human and blood is the source of the nucleic acid. However, any bodily fluid that contains suitable nucleic acid specimens is contemplated, including lymph, saliva, urine, or other bodily excretions. Alternatively, the nucleic acid could be derived from soft tissue, hair, or bone. When methods of obtaining nucleic acid from a human and determining whether the human has the novel TS SNP and/or the 3′ UTR polymorphism are utilized, it is preferable that the nucleic acid is amplified and sequenced using methods well known in the genetic arts, such as the PCR methods discussed throughout this disclosure. Another preferable embodiment of the present invention uses high throughput screening methods to test multiple samples at the same time. Typically in high throughput screening, the nucleic acid molecules to be tested are bound to a solid support, such as a microtiter dish, amplified and labeled, and the results read by a machine adapted to such use.

Once the TS SNP and/or the 3′ UTR polymorphism is screened for and has been identified or found absent in a particular patient, other methods of the invention are prognostic and diagnostic methods that provide for the indication of whether a patient will be a good candidate for chemotherapeutic and/or anti-CVD drugs. It has been found that high levels of TS transcription are linked to a shorter survival rate as compared to those patients with a lower TS transcription rate (Ulrich et al., 2002). With respect to the relationship between TS and cardiovascular disease, TS and the enzyme 5,10-methylenetetrahydrofolate reductase compete for limited supplies of folate required for the remethylation of homocysteine, an amino acid found in blood. Elevated levels homocysteine are used to identify patients at increased risk of CVD (Trinh et al., 2002). Thus, the relationship between TS and cancer and, independently, TS and CVD make the TS SNP a valuable tool for screening for (1) the likelihood that a given individual will develop cancer or CVD, (2) the potential severity of the relevant disease, and (3) treatments that are more likely to work given the form of the TS gene.

For example, if a patient has two copies of the 3R wild-type form of the TS gene (3R/3R), then there are two USF E-boxes per allele and the transcription of TS in that person will likely be higher than a person with 3R/3RV, 2R/2R, 2R/3R, or 2R/3RV TS alleles. A physician would then try to design a useful therapy for that person, knowing that TS expression is high. Useful therapies might include targeting the TS gene to reduce TS expression and/or targeting another aspect of the disease to offset the high level of TS produced. If a patient were screened and found not to have a high level of TS transcription, then a physician might decide to go with the conventional treatment, which has markedly higher success rates in patients without high TS transcription. There are many possible ways to use the presence or absence of the TS SNP in conjunction with the knowledge of the tandem repeats because the presence of the TS SNP means that an extra copy of the tandem repeat does not confer higher TS transcriptional activity. Thus, those skilled in the art will be better able to genetically screen a person and accurately determine the level of TS transcription from the screen alone. Then, the novel TS diagnostic marker can then be translated into preferred methods of treatment for the given disease.

A separate aspect of the present invention focuses on another polymorphism—a 6 bp/1494 deletion polymorphism in the 3′-untranslated region (3′UTR) of the human TS gene. The present invention discovered that this polymorphism causes message instability and is associated with decreased intratumoral TS mRNA levels. Insertion of the 3′UTR of TS containing the +6 bp/1494 polymorphism into the luciferase 3′UTR resulted in a ˜35% decrease in luciferase activity, and a similar decrease in mRNA levels, compared to the empty pGL3-control vector. A series of deletions of the 3′UTR of TS resulted in no significant differences in luciferase activity compared to the full-length 3′UTR, showing that regions within the TS-3′UTR are relatively stable overall. Insertion of the TS-3′UTR containing the −6 bp/1494 deletion polymorphism resulted in a 70% decrease in luciferase activity and a 60% decrease in mRNA levels compared to the empty pGL3-control vector, indicating that the deletion polymorphism caused a decrease in mRNA stability.

Further proving that the deletion causes instability, the TS-3′UTR containing the −6 bp/1494 deletion polymorphism had a significantly higher rate of message degradation compared to the +6 bp/1494 construct. Measurement of intratumoral TS mRNA levels demonstrated that individuals homozygous for the insertion (+6 bp/+6 bp) polymorphism had significantly higher TS mRNA levels compared to individuals that were homozygous for the deletion (−6 bp/−6 bp) polymorphism (p<0.007). Statistical analysis determined the frequency of the −6 bp/1494 deletion polymorphism in a variety of ethnic populations to be 41% in non-Hispanic whites, 26% in Hispanic whites and 52% in African-Americans. Taken together, these results signify that the −6 bp/1494 deletion polymorphism in the 3′UTR of TS is associated with decreased mRNA stability in vitro, lower intratumoral TS expression in vivo.

Thus, knowledge of the effect of the −6 bp/1494 deletion polymorphism is a useful screening tool in predicting an individual's TS mRNA levels in a clinical setting. Because the −6 bp/1494 deletion polymorphism causes TS mRNA instability, a screen can be used to find individuals with this deletion polymorphism. The results of the screen can then be used to tailor cancer treatments and/or cancer prevention therapies depending on the result. For example, as explained above, an individual with the deletion polymorphism has less stable TS and so is more likely to be responsive to therapies that target TS. Further, an individual with the deletion polymorphism has a lesser chance of developing cancer because there is less probability that the TS in the cancerous cells will be stable and be capable of robustly progressing and possibly spreading throughout the individual.

The polymorphisms of the present invention may be used separately as screens, but are preferably used together to determine whether individuals have a higher likelihood of TS disruption (3R/3RV, 2R/2R, 2R/3R, or 2R/3RV with −6 bp/1494 deletion), average likelihood of TS disruption (3R/3RV, 2R/2R, 2R/3R, or 2R/3RV with +6 bp/1494, or (3R/3R with −6 bp/1494 deletion), or lower likelihood of TS disruption (3R/3R with +6 bp/1494). Using the polymorphisms in conjunction may allow a diagnostician a more precise estimate of TS disruption and thus, a more accurate idea of how that individual with either respond to cancer treatment or cardiovascular disease treatment.

Drug selection is also linked to the polymorphisms because it can be investigated how a particular drug acts within the portion of the population possessing a particular TS polymorphism. If the higher TS transcription and/or activity is known to be associated with the reduced efficacy of a given drug, a clinician pursue other methods of treating the cancer and/or CVD, either instead of or in addition to the administration of the pharmaceutical. Conversely, if lower TS transcription and/or activity is known to be associated with the increased efficacy of a given pharmaceutical, it would likely be advantageous to administer that pharmaceutical to a person matching the reduced TS profile. The methods further indicates how likely it is that an individual with develop cancer and/or cardiovascular disease based on the probable transcription rate and stability of TS. Again, the greater the stability of TS, the higher the likelihood of developing cancer and/or cardiovascular disease and lower the likelihood of effective treatment of those diseases because stable TS allows for the survival and propagation of the diseased tissue.

A further aspect of the invention are kits for carrying out the methods of TS polymorphism identification and screening described herein. Preferably, the kits will comprise primers, probes, implements for the arrays, screening arrays, and instructions for use. Preferably, the kits also contain the reagents, polymerase, tubes, and any other substance or equipment required to carry out the identification of one or both of the polymorphisms.

Other aspects of the present invention contemplate using genetic and or protein based manipulation to control the TS transcription and or TS enzyme activity. If genetic manipulation is intended, vectors containing the preferred form of the TS gene may be introduced in vitro or in vivo to cells of the individual. Alternatively, host cells may be genetically engineered with vectors of the invention and produce the polypeptides of the invention by recombinant techniques both in vitro and in vivo, as well as ex vivo procedures. Introduction of the TS polynucleotides with the preferred polymorphisms into host cells can then be effected by methods described in many standard laboratory manuals (See Davis et al., Basic Methods In Molecular Biology (1986) and Sambrook et al., Molecular Cloning: A Laboratory Manual, 2nd Ed., Cold Spring Harbor Laboratory Press, Cold Spring Harbor, N.Y. (1989)). The use of vectors, preferably targeted recombinant viral vectors, is well known in the art (See, for example, U.S. Pat. No. 6,635,476).

The vectors should incorporate relevant promoters, enhancers, and the like to aid the alteration of the TS sequence. Promoter regions can be selected from any desired gene with selectable markers. Two appropriate vectors are pKK232-8 and pCM7. Particular named bacterial promoters include lacI, lacZ, T3, T7, gpt, lambda PR, PL and trp. Eukaryotic promoters include CMV immediate early, HSV thymidine kinase, early and late SV40, LTRs from retrovirus, and mouse metallothionein-1. Selection of the appropriate vector and promoter is well within the level of ordinary skill in the art.

A further aspect of the invention is the use of antibodies to the TS enzymes to reduce the activity of the TS enzyme. Preferably, the antibodies are targeted and immunospecifically bind to the TS enzymes with the highest TS activity. Polyclonal or monoclonal antibodies directed towards the polypeptide encoded by TS may be prepared according to standard methods. Monoclonal antibodies may be prepared according to general hybridoma methods of Kohler and Milstein, Nature (1975) 256:495-497), the trioma technique, the human B-cell hybridoma technique (Kozbor et al., Immunology Today (1983) 4:72) and the EBV-hybridoma technique (Cole et al., Monoclonal Antibodies And Cancer Therapy, pp. 77-96, Alan R. Liss, Inc., 1985). Antibodies utilized in the present invention may be polyclonal antibodies, although monoclonal antibodies are preferred because they may be reproduced by cell culture or recombinantly, and may be modified to reduce their antigenicity. Polyclonal antibodies may be raised by a standard protocol by injecting a production animal with an antigenic composition, formulated as described above. (See, e.g., Harlow and Lane, Antibodies: A Laboratory Manual, Cold Spring Harbor Laboratory, 1988.)

Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. The immortal cell line utilized is preferably selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT). The antibodies may be administered parenterally, intravenously, or orally.

These and other embodiments of the inventions will be apparent from the description of the experiments.

II. Experiments

Overview of the 5′ SNP Experiment Series

The following examples are meant to illustrate the present invention and are not limitations upon it. All citations throughout the disclosure are incorporated by reference and found in a complete listing at the end of the written description.

Thymidylate synthase (TS) gene expression is modulated in part by a polymorphism in the 5′ regulatory region of the gene. The polymorphism consists of either two repeats (2R) or three repeats (3R) of a 28 bp sequence, yielding greater TS gene expression and protein levels with a 3R genotype. The sequence of the third repeat is a 28 base pair sequence of CCGCGCCACTTGGCCTGCCTCCGTCCCG, designated SEQ ID NO:1. Two USF family E-box consensus elements are found within the tandem repeats of the 3R genotype and one within the 2R genotype. These elements bind USF protein complexes in vitro by electrophoretic mobility shift assays (EMSA) and in vivo by ChIP assay. The present disclosure shows that the additional USF consensus element within the 3R construct confers greater transcriptional activity relative to the 2R construct. The present invention demonstrates that mutagenesis of the USF sites shows that the transcriptional regulation of TS is dependent on USF proteins binding within the tandem repeats.

The identification of a novel G→C single nucleotide polymorphism in the second repeat of 3R alleles within the USF consensus element alters the ability of USF proteins to bind to the mutated site, and thus alters the transcriptional activation of TS genes bearing this genotype. Through RFLP analysis, the frequency of this polymorphism (3RV) was determined to be 56% of all 3R alleles in healthy Non-Hispanic White individuals. A single nucleotide polymorphism is a DNA sequence variations that occurs when a single nucleotide (A, T, C, or G) in the genome sequence is changed. Screening for the SNP in combination with the tandem repeat polymorphism significantly increases the value of the tandem repeats alone in predicting response and survival to 5-FU/LV chemotherapy treatment. The more non-variant 3R copies of the TS allele that a subject has, the worse the response to chemotherapy drugs. Therefore, this novel SNP of the 5′ tandem repeat polymorphism can be used as a predictor of clinical outcome to thymidylate synthase inhibitors and other chemotherapeutic agents.

Thus, the present invention characterizes the mechanism of transcriptional activation from the tandem repeats and describes how an additional 28 bp repeat can enhance transcriptional activity. The present invention also identifies a highly penetrant single nucleotide polymorphism within the 3R that can abolish its increased transcriptional activity relative to the 2R, and show that sequence variations within the tandem repeats have functional significance.

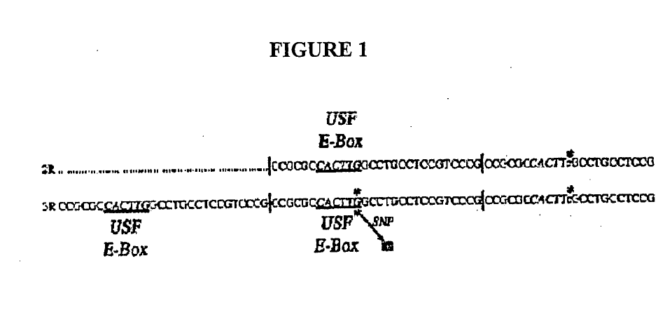

In order to determine what regulatory factors were involved in transcriptional activation from the tandem repeats, sequence consensus elements within the 28 base pair regions were investigated. An E-box site, CACTTG, lies within the middle of the first and second repeats of the 3R polymorphism and within the first repeat of the 2R polymorphism (See FIG. 1). EMSA analysis using nuclear extracts with competitor oligonucleotides identified USF complexes bound to this element in vitro. However, only USF-1 that had been phosphorylated by cdc2/p34 was bound to its consensus element within the tandem repeats. CHIP analysis shows the presence of both USF-1 and USF-2 at the TS 5′ regulatory region in vivo. The region amplified in the ChIP assay contained only the putative E-box sites located within the tandem repeats and shows that USF-1 and USF-2 were bound to these sites in vivo.

Through site-directed mutagenesis, it is shown that USF consensus elements within the first repeat of either the 2R or 3R constructs are necessary for efficient transcriptional activation of the luciferase reporter gene constructs. These assays also showed that an extra repeat in the 3R construct adds an additional USF binding site that leads to increased transcriptional activity compared to the 2R construct, in the absence and presence of exogenous USF-1. Thus, the enhancer function of the tandem repeats at the transcriptional level increases as the number of USF E-box sites increase. Although USF-2 did not activate the TS promoter constructs significantly, the presence of USF-2 in the ChIP assay, and the fact that these proteins exist as heterodimers to a large extent in the cell, suggests that USF-2 may be present in complex with USF-1 at the TS tandem repeats in vivo

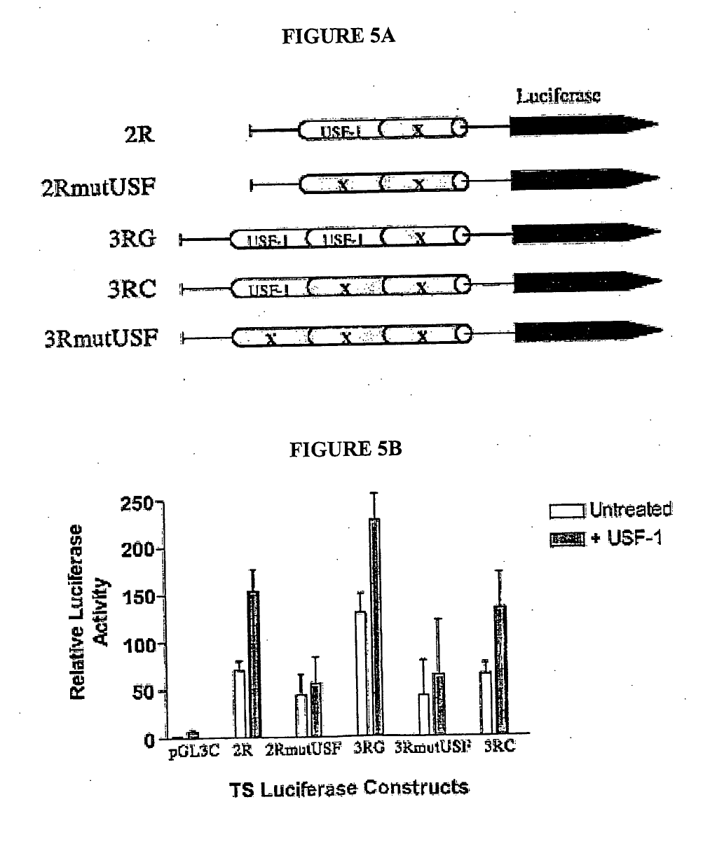

It has been postulated that the number of tandem repeats in the TS gene itself determines the level of TS expression. However, the present invention modifies this theory with the identification of an unexpected and novel SNP within the tandem repeats that alters the enhancer function of an extra repeat. A single G→C base change found at the 12^{th} nucleotide of the second repeat in the 3R genotype, alters a critical residue in the USF consensus element. Thus, it is not the number of tandem repeats alone, but the number of functional tandem repeats that determines the level of TS transcription. An EMSA assay shows that this base change abolishes the ability of USF complexes to bind within the repeat and effectively eliminates the E-box site. A 3R construct bearing this variation, 3RV, was isolated from patient genomic DNA and used in luciferase reporter assays to analyze the effects of this polymorphism on transcription. The 3RV construct displayed a similar transcriptional activity as a 2R construct (FIG. 5B). These results suggest that the addition of a 28 bp repeat alone is not sufficient for enhanced transcriptional activity of the TS gene, but that a USF E-box element is required within the extra repeat in order to enhance transcription.

This experiment revised the previous PCR based method for determining tandem repeat polymorphism genotype (Horie et al., 1995) into a restriction fragment length polymorphism (RFLP) technique that includes a screen for the G→C SNP. A smaller PCR fragment is amplified (to remove extraneous HaeIII sites) and half of the sample is left undigested while the other half is digested with the HaeIII restriction enzyme. When patient samples are run side-by-side on an agarose gel, the tandem repeat polymorphism as well as the SNP can be determined for both alleles. The frequency of the SNP in 99 colorectal cancer patients (Table 1) was determined using this novel method.

TABLE 1
Distribution of the 5′-TS tandem repeat polymorphism
and the novel G→C polymorphism in the second repeat
of the 3R among 99 Non-Hispanic White individuals

| | | | 2R/ | 3R/ | 3R/ | 3RV/ | |
|---|---|---|---|---|---|---|---|
| Genotype | 2R/2R | 2R/3R | 3RV | 3R | 3RV | 3RV | Total |
| Number | 19 | 13 | 31 | 11 | 16 | 9 | 99 |
| Allele | | | | | | | Frequency |
| 2R-Allele | 38 | 13 | 31 | — | — | — | .414¹ |
| 3R-Allele | — | 13 | 31 | 22 | 32 | 18 | .586¹ |
| 3RV-Allele | — | — | 31 | — | 16 | 18 | .560² |

¹Frequency of allele is shown as percentage of all alleles.
²Frequency of allele is shown as percentage of 3R alleles only.

The SNP is easily screened for with the addition of a simple restriction digestion and generates useful information for clinicians in order to tailor individual chemotherapy in respect to both tumor response and host toxicity in relation to cancer treatment. In relation to CVD treatment, clinicians can determine if a subject is at a higher risk for CVD by looking at the number of non-variant alleles. The clinician can then tailor the therapy accordingly, noting that the levels of folate will likely be lower and homocysteine will likely be higher than in subject with more 2R and/or 3RV alleles, thus making that individual more susceptible to CVD.

The regulation and functions of USF proteins add further complexity to the TS-inhibition pathway and to the formation and progression of carcinogenesis. The USF proteins have been traditionally described as ubiquitous regulatory factors but recent evidence has shown that these proteins can be misregulated in some forms of cancer (Kawakami et al., 1999) and are overexpressed during periods of malnutrition, particularly protein-free diets (Matsukawa et al., 2001). Further, USF-1 is activated by the stress-responsive p38 kinase. It has been postulated that this activation provides a link between stress stimuli and the subsequent changes in gene expression that occur as a result of treatment with stress-inducing agents (Galibert et al., 2001), possibly including chemotherapeutic agents. Thus, it can be hypothesized that overexpression of USF proteins could cause increased activation of genes targeted by USF-1/USF-2 complexes, thereby implicating the USF proteins as mediators of TS overexpression in vivo. USF overexpression could also lead to TS overexpression indirectly, through activation of the tumor-suppressor p53 (Reisman et al., 1993), which transactivates the TS promoter. Based on this evidence, USF-1 and USF-2 could play a role in causing the drug resistance seen in patients treated with TS inhibitors through direct and indirect mechanisms. The present invention contemplates blocking USF binding sites on the TS gene so that, even if USF were overexpressed, the TS E-boxes would be competitively bound by a non-TS activating substance.

Conversely, loss of USF function could contribute to carcinogenesis (Ismail et al., 1999). Genetic alterations in APC are thought to be one of the earliest steps in colon carcinogenesis (Ichii et al., 1992) and loss of APC gene function has been correlated with increased c-Myc oncogene activity (Erisman et al., 1985; Jaiswal et al., 1999). USF-1/USF-2 complexes have been shown to transactivate the APC gene (Jaiswal et al., 2001). Since USF proteins antagonize the effects of c-Myc (Luo et al., 1996), it has been proposed that loss of USF function could cause down regulation of APC leading to increased c-Myc expression and enhanced cellular proliferation (Pullarkat et al., 2001).

Here, the present invention provides evidence for a direct role of USF proteins in the regulation of TS gene expression and suggests that the inhibition of USF activity or USF binding sites could also be considered as a modulating therapy for TS-directed anti-cancer drugs. Based on the results, the fact that the novel SNP of the present invention alters the ability of the repeats to function as enhancers of transcription explains discrepancies in response to 5-FU treatment. Considering the importance of the TS reaction in folate metabolism, the novel polymorphism may have additional influence in the modulation of other folate dependent pathways. In addition to thymidylate biosynthesis, purine synthesis, methionine regeneration, and other one-carbon donor reactions, such as those involved in DNA methylation, are all influenced by this polymorphism. Here, the present invention demonstrates that a transcriptional component within the tandem repeats exists and proves that this component is altered by differences in the nucleotide sequence of the repeats. The present comprehensive analysis of both polymorphisms contributes to a more precise prediction of TS gene expression and clinical outcome to fluoropyrimidines and other chemotherapeutic drugs, and to predicting and treating CVD.

1. The 28 bp Tandem Repeats in the 5′ Regulatory Region of the Human TS Gene are not Identical in Their Nucleotide Sequences

The published sequence of the human TS gene and its 5′ upstream regions (Takeishi et al., 1989) shows that there are two single base changes in the last 28 bp repeat of both the 2R and 3R genotypes, and recent evidence has shown that these sequence differences exist in the last repeats of the 4R and 5R alleles as well (Luo et al., 2002). The consequences of these base changes on TS gene expression, as well as the frequency of these base changes, have not been examined. Thus, this experiment sought to verify the presence of sequence differences within the repeats, and to look for other base changes and potential polymorphisms. By direct sequencing of 14 human genomic DNA samples, the experiment verified the presence of the two base changes in the last repeats of 2R and 3R, and identified a novel single nucleotide polymorphism within the second repeat of 3R (FIG. 1, asterisks).

FIG. 1 is the structure of a tandem repeat polymorphism within the 5′-untranslated region of the human TS gene. An enhancer polymorphism in the 5′-untranslated region of the thymidylate synthase gene consists of either two or three 28 bp repeats. The third repeat is SEQ ID NO: 1. The nucleotide sequence of these repeats is shown above and bears variations within each repeat. A putative E-box binding site for upstream stimulatory factor (USF-1/USF-2) has been identified and is underlined and bolded within each repeat. The consensus sequence for USF DNA binding is CANNTG (where N is any nucleotide). Repeats one and two of 3R and repeat one of 2R, contain USF consensus elements (underlined) while the last repeat in either construct contain an imperfect or variant consensus sequence due to a G→C base change (asterisks) that disrupts the putative E-box. The last nucleotide of the final repeat in 2R and 3R also bears a G→C base change.

In order to determine if these base changes exert a functional role on gene expression, it was first sought to identify regulatory factors that bind within the 28 bp TS tandem repeats. Both the 28 bp sequence lacking and the 28 bp sequence bearing the base changes were scanned for putative transcription factor binding sites using the TRANSFAC database (Wingender et al., 2000). A USF E-box consensus element (CACTTG) was found within the first repeat of the 2R genotype and within the first two repeats of the 3R genotype, but not in the last repeat of either genotype (FIG. 1). The C at the 12^{th} nucleotide of the last repeat of 2R and 3R lies within the USF consensus sequence element at a critical nucleotide for USF binding. The potential SNP at the 12^{th} nucleotide in the second repeat of 3R changed the USF consensus element in a similar fashion (FIG. 1, shaded nucleotide). These results show that USF regulatory factors can bind to sequences within the TS tandem repeats.

2. Phospho-USF-1 Binds to Consensus Elements Within the TS Tandem Repeats but not to Repeats Containing the G→C Base Change at the 12th Nucleotide

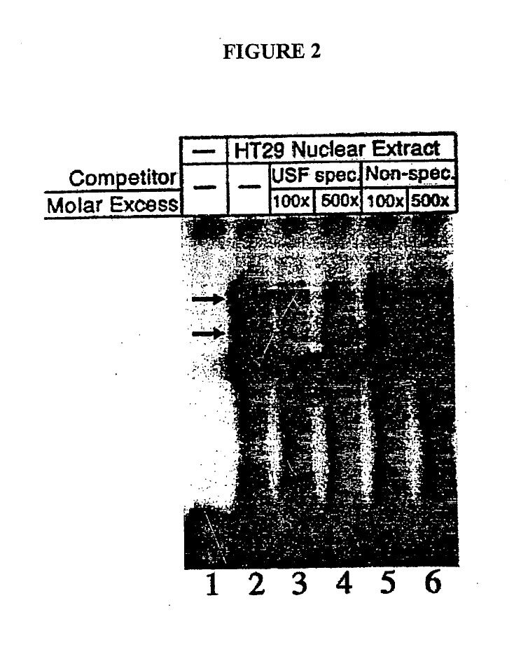

To determine the sequence-specific binding of USF proteins to the TS tandem repeats in vitro, a 28 bp sequence bearing the putative USF consensus E-box element was used as a probe in electrophoretic mobility shift assays (EMSA). FIG. 2 is a EMSA showing that USF proteins bind to the E-box site within the tandem repeats of the human TS gene in HT29 nuclear extracts. Gel mobility shift analyses were performed using HT29 nuclear extracts with a ³²P-labeled 28 bp probe corresponding to a tandem repeat sequence containing an intact E-box site. In FIG. 2, lane 0.1 is free probe. In lane 2, 2.5 μg of HT29 nuclear extracts were incubated with probe in the absence of unlabeled competitor oligonucleotide, resulting in the presence of numerous band shifts on the gel.

The addition of an unlabeled specific USF competitor oligonucleotide to the reaction resulted in the absence of two bands, which were again present when non-specific competitor was added (FIG. 2, lanes 3-6). In lanes 3-4, extracts were pre-incubated with unlabeled specific competitor oligonucleotides to USF-1 in increasing molar excess. In lanes 5-6, extracts were pre-incubated with unlabeled non-specific competitor poly (dIdC) in increasing molar excess. Arrows indicate USF protein complexes. These competition experiments show sequence-specific binding of USF complexes to the 28 bp tandem repeat sequences containing intact USF E-box elements.

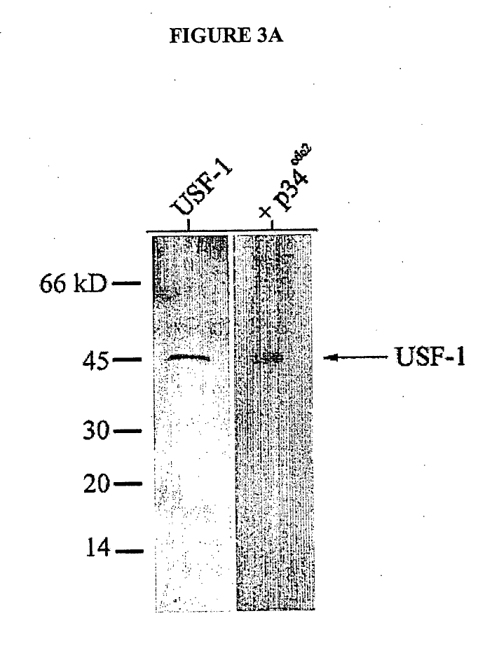

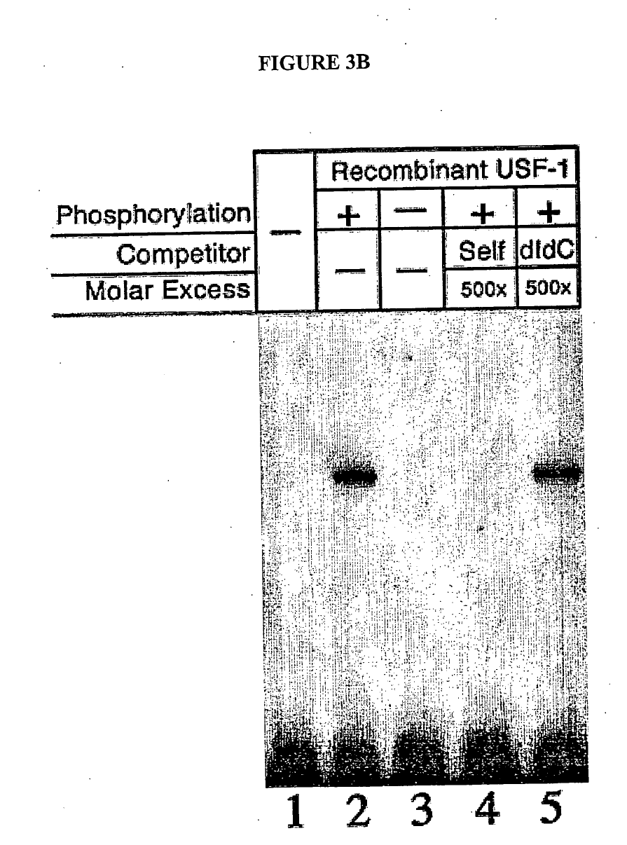

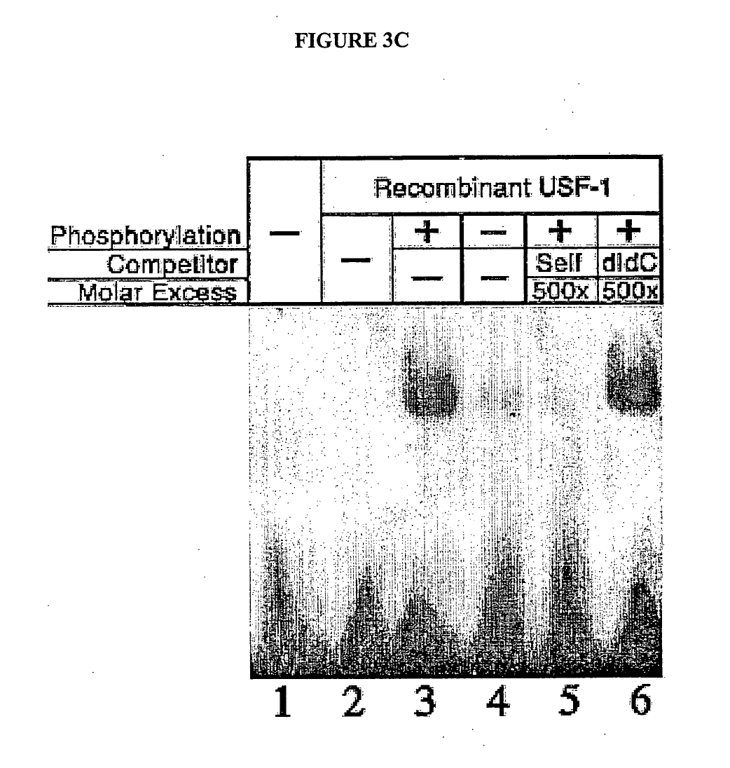

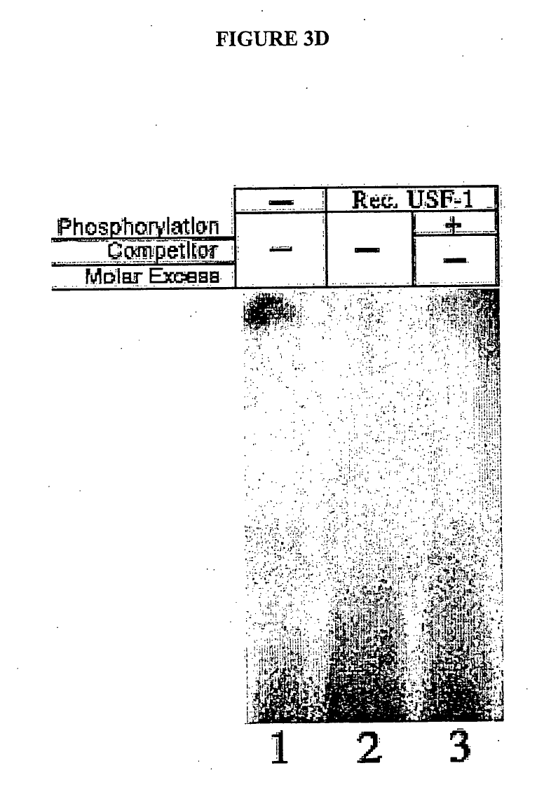

Since USF-1 shows increased affinity for its DNA consensus element when it is phosphorylated, the ability of the phosphorylated and unphosphorylated forms of USF-1 to bind to the putative consensus element within the 28 bp repeat sequence was tested. Recombinant USF-1 was expressed in E. coli with a 6-histidine tag and purified on a Ni-NTA column. Cdc2/p34 was immunoprecipitated from HeLa S3 cells and used in an in vitro kinase reaction to phosphorylate 200 ng of recombinant USF-1 (FIG. 3A). ³²P-labeled ATP was used in the control reaction for visualization of phosphorylation after exposure of the film (right panel).

When both forms of USF-1 were used in an EMSA assay utilizing the perfect USF-1 consensus element as a probe, only the phosphorylated form was able to bind (FIG. 3B, lanes 2 and 3). Gel mobility shift analyses were performed using recombinant USF-1 with a ³²P-labeled USF-1 specific consensus probe containing an intact E-box site. Lane 1 contained only free probe. Lane 2 was 30 ng of recombinant phospho-USF-1 incubated with probe in the absence of unlabeled competitor oligonucleotides. Lane 3 contained 30 ng of recombinant unphosphorylated USF-1 incubated with probe in the absence of unlabeled specific competitor oligonucleotides. In lanes 4-5, phospho-USF-1 was pre-incubated with 500 molar excess of unlabeled USF-1 specific competitor oligonucleotide and 500 molar excess of nonspecific dIdC competitor, respectively.

To determine the ability of the phosphorylated form of USF-1 to bind to its consensus element within the TS repeat, an EMSA assay was carried out using the ³²P-labeled 28 bp sequence as a probe corresponding to one tandem repeat containing an intact E-box site. Lane 1 was free probe. In lane 2, 30 ng of recombinant USF-1 was incubated with probe in the absence of unlabeled competitor oligonucleotides. In lane 3, 30 ng of phospho-USF-1 was incubated with probe in the absence of unlabeled competitor oligonucleotides. In lanes 4-6, phospho-USF-1 was pre-incubated with 500 molar excess of: unlabeled probe, USF-1 specific competitor, and non-specific poly (dIdC) competitor oligonucleotides, respectively. Incubation of the phosphorylated form of USF-1 with the probe caused a shift on the gel that was abolished by the addition of unlabeled specific competitor oligonucleotides (FIG. 3C, lanes 3 and 5). This data further proves that only the phosphorylated form of USF-1 can bind its consensus element within the tandem repeats.

Since the potential G→C SNP at the 12^{th} nucleotide of the 28 bp repeats lies within the USF binding site, the ability of the recombinant USF-1 protein to bind the variant consensus element by EMSA was tested. Neither the unphosphorylated or phosphorylated forms of USF-1 showed any affinity to this variant sequence (FIG. 3D, right panel). Gel mobility shift analyses were performed using recombinant USF-1 with a ³²P-labeled 28 bp probe corresponding to one tandem repeat containing a G→C base change at the 12^{th} nucleotide. Lane 1 was free probe. In lane 2, 30 ng of recombinant USF-1 was incubated with probe. In lane 3, 30 ng of phospho-USF-1 was incubated with probe. This data shows that the potential SNP within the tandem repeats abolishes USF binding by disrupting the USF consensus E-box element.

3. USF-1 and USF-2 Bind to the Thymidylate Synthase Tandem Repeats In Vivo

The results of the in vitro assays show sequence-specific binding of USF-1 and USF-2 to the tandem repeats of the thymidylate synthase gene at E-box consensus sites. To determine if USF-1 and USF-2 were bound to these elements in vivo, a chromatin immunoprecipitation (ChIP) assay using live 293 (human embryonic kidney) cells was performed using genomic DNA from 1×10⁶ antibodies for USF-1 and USF-2. Input DNA was a 20 μl aliquot of DNA taken before addition of antibodies and the no-antibody control was performed along side USF-1 and USF-2 immunoprecipitations without the addition of antibody. After formaldehyde cross-linking of proteins to DNA and shearing of genomic DNA by sonication, immunoprecipitations using USF-1 and USF-2 antibodies were performed. The immunoprecipitations included a control reaction, which was performed without the antibodies.

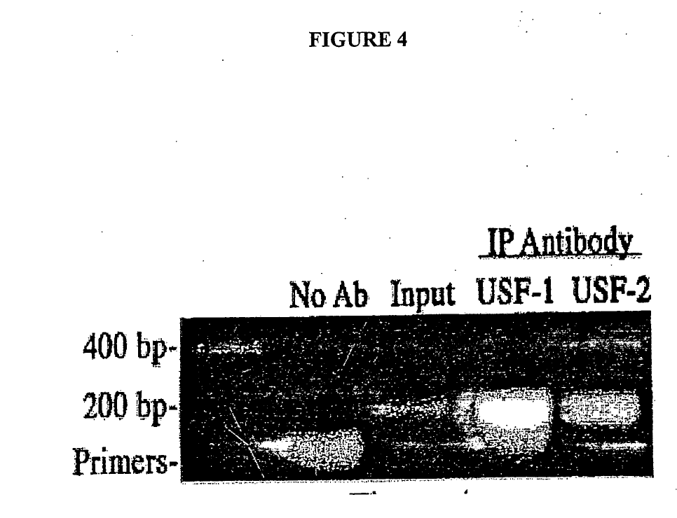

After the pull downs, PCR amplification was performed at 64.8° C. to determine if the TS 5′ regulatory region containing the tandem repeats (+15 to +195 relative to the transcription start site) was bound by USF-1 or USF-2. The PCR product was then ethanol precipitated and electrophoresed on a 1.5% agarose gel. The 180 bp fragment was amplified from the immunoprecipitations using USF-1 and USF-2 polyclonal antibodies but was not present in the control reaction lacking antibody (FIG. 4). These results show the presence of USF-1 and USF-2 on the chromatin at the TS locus, which includes the tandem repeats and E-box elements. This particular region of DNA contains no other putative E-box elements other than those located within the tandem repeats. The presence of USF-1 and USF-2 at the TS 5′ regulatory region showed that these proteins bind to the E-box elements located within the tandem repeats. These data led to the examination of the potential role of these proteins in activating transcription of TS 5′ regulatory region reporter constructs.

4. USF-1 Transactivates the TS Promoter Through Binding of Tandem Repeats Containing E-Box Elements

To examine the ability of USF-1 and USF-2 to enhance transcription through binding within the tandem repeats, the 5′ promoter region of the human TS gene from −313 to +195 (relative to TS transcription start), including the 5′ untranslated region, was cloned into the TATA-less pGL3-Basic luciferase reporter vector just upstream of the luciferase translation start site. Both 2R and 3R constructs were individually cloned into the vector and 2RmutUSF and 3RmutUSF were created by altering the indicated USF consensus elements through site-directed mutagenesis. FIG. 5A is a diagram of those two TS luciferase reporter constructs. The 3RV construct lacks an E-box element in the second repeat due to a G→C SNP polymorphism. All E-box elements are labeled USF and all variant or mutant elements are labeled with an X.

These constructs were co-transfected into 293 cells along with either a USF-1 expressing vector, a USF-2 expressing vector, or an empty vector. Results from these experiments show that there was an increase in relative luciferase activity from both the 2R and 3R constructs in the presence of USF-1 (FIG. 5B). This 2-3 fold increase in transcriptional activity is consistent with previous reports of activation by USF. USF-2 activation led to a modest increase in relative luciferase activity. The 3R construct had greater luciferase activity than the 2R construct in both the absence and presence of exogenous USF-1 protein expression and this difference between 2R and 3R transcriptional activity is consistent with previous reports in a similar luciferase system. These differences are significant because subtle differences in TS gene expression have been shown to be significant in predicting response to 5-FU in vivo (Lenz et al., 1996). Both 2RmutUSF and 3RmutUSF showed dramatically decreased transcriptional activity below endogenous levels of transcription, compared to their wild-type counterparts, indicating that these USF sites, one in the 2R and two in the 3R, are critical to TS promoter activation. Consequently, these sites may be responsible for greater transcriptional activity from the 3R overall.

Since the single G→C base change at the 12^{th} nucleotide of the 28 bp repeats can abolish the ability of USF proteins to bind to this site by EMSA, it was desirable to determine whether this base change would alter the ability of USF-1 to transactivate the 3R TS promoter construct. The 3R variant (3RV) reporter construct (FIG. 5A) had decreased transcriptional activity compared to the 3R, in the absence and presence of exogenous USF-1. In addition, 3RV had a similar ability to transactivate the luciferase reporter gene as the 2R construct (FIG. 5B). These data show that the ability of the tandem repeats to enhance transcription increases only as the number of USF consensus elements increase, and not necessarily as tandem repeats increase. Hence, the potential SNP within the second repeat of 3R is a determinant of the ability of the 3R construct to act as an enhancer of transcription, relative to the 2R construct. Overall, USF-2 activation led to a modest increase in relative luciferase activity alone, in USF-1 and USF-2 co-transfections showed no increase in luciferase activity compared to USF-1 alone.

5. Characterization of a Novel Single-Nucleotide Polymorphism (SNP) by Restriction Fragment Length Polymorphism (RFLP) Analysis

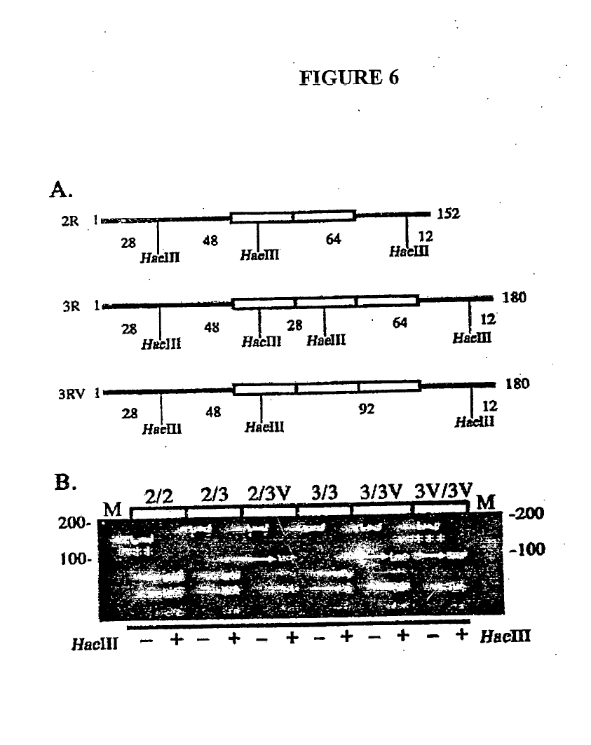

To determine the frequency of the potential SNP in a large population, an RFLP analysis was developed. FIG. 6A is a diagram of the HaeIII restriction map of the TS tandem repeat fragments produced in this RFLP analysis. This map shows the HaeIII restriction endonuclease sites within the fragments produced by polymerase chain reaction (PCR) for the RFLP analysis.

PCR was carried out using genomic DNA samples from 99 healthy Non-Hispanic White individuals, yielding PCR fragments of 213 bp for 2R alleles, 241 bp for 3R alleles and both fragments for 2R/3R heterozygotes. TS genotypes could be obtained from 99 samples. The G→C base change in the 3RV removes a HaeIII restriction endonuclease site and changes the banding pattern of the digested PCR fragment on a 3% sea-plaque agarose gel in 0.5×TBE (FIG. 6B, undigested samples). Half of the PCR products were digested with the HaeIII restriction enzyme and half were left undigested. The arrows point to the additional 92 bp fragment that is present in wild type samples, but is absent in samples positive for the G→C polymorphism. Genotypes are listed above corresponding lanes showing repeat polymorphism (2 or 3) and G→C SNP polymorphism (V for variant).

Digested and undigested PCR products from each patient were run in adjacent lanes to determine the repeat polymorphism genotypes and the G→C SNP genotypes of each allele. Running undigested product next to digested product was necessary since there are similar banding patterns for 2R/2R, 2R/3R, and 3R/3R as well as for 2R/3RV and 3R/3RV when they are digested with the enzyme. In some samples, non-specific DNA product was observed at ˜100 bp in length in the undigested samples. This non-specific DNA resulted in the presence of a ˜60 bp band in the HaeIII digested samples that did not interfere with interpretation of the genotype. Nevertheless, a single PCR reaction followed by digestion of half the sample with HaeIII, yielded patient genotypes for the tandem repeat polymorphism and the SNP within the tandem repeats.

The G→C SNP at the 12^{th} nucleotide was observed only in the second repeat of 3R genotypes. The frequency of the 3R among the 100 Caucasian individuals was 58.6% and consistent with earlier reports in Caucasians. The frequency of the novel G→C SNP at the 12^{th} nucleotide in the second repeat of the 3R was 56% among all 3R carriers. This data suggests that the G→C base change at the 12^{th} nucleotide of the second repeat of 3R alleles is a highly penetrant polymorphism among Non-Hispanic Whites.

6. The SNP Significantly Increases the Value of the TS Tandem Repeats in Predicting Response and Survival to 5-FU/LV in Colorectal Cancer

To explore the role of SNP as a predictive marker, 40 patients were evaluated with disseminated colorectal cancer (SWOG 9420 and 3C-92-2) for response and survival to protracted infusion of 5-fluorouracil. The distribution of the TS tandem repeat polymorphism was as follows: 2R/2R 20% (8/40), 2R/3R 50% (20/40), and 3R/3R 30% (12/40). Patients confirmed for the 2R/2R genotype had a 50% (4/8) response to 5-FU as compared to 15% (3/20) in the 2R/3R group. No patient with a 3R/3R genotype showed disease response (0/12). However, this association did not reach statistical significance (P=0.089, Fisher's Exact test). Patients possessing the 2R/2R genotype showed a median survival of 16.2 months compared to 7.4 months in the heterozygous group and 8.4 months for 3R/3R carriers, respectively. This relationship also lacked statistical significant (P=0.14, Logrank Test).

Patient samples were re-classified into two groups based on predicted high and low TS expression using the tandem repeat polymorphism and the SNP. Since it was hypothesized that the SNP, or variant 3R (3RV) allele, would decrease the TS gene expression of a 3R allele, 21 patients were grouped with genotypes of 2R/2R, 2R/3RV and 3RV/3RV into the predicted “low TS expression” group (Group A) and 19 patients with 2R/3R, 3R/3RV, and 3R/3R into the predicted “high TS expression” group (Group B). These groups were then re-evaluated for an association between TS genotypes and clinical outcome to 5-FU chemotherapy.

Patients possessing one of the low-expression TS genotypes showed an improved response rate to chemotherapy. Thirty-three percent (33%) (7/21) of patients in Group A showed disease response, compared to 0% (0/19) of the patients in Group B. Sixty-three percent (63%) (12/19) of patients in Group B showed disease progression compared to only 48% (10/21) in Group A (P=0.019, Fisher's Exact Test) (Table: 2). In addition, study participants of Group A demonstrated a superior survival of 10.1 months compared to only 7.4 months for patients of Group B (P=0.035, Logrank Test).

TABLE 2
Association between TS genotypes and clinical outcome to 5-FU for
colorectal cancer patients

| | Clinical Response | | |
|---|---|---|---|
| | | Stable | |
| TS-Genotype | Response | Disease | Progression |
| 2R/2R (n = 8) | 4 (50%) | 1 (13%) | 3 (37%) |
| 2R/3R (n = 20) | 3 (15%) | 5 (25%) | 12 (60%) |
| 3R/3R (n = 12) | 0 (0%) | 5 (42%) | 7 (58%) |
| P = 0.089¹ | | | |
| 2R/2R | 7 (33%) | 4 (19%) | 10 (48%) |
| 2R/3RV | | | |
| 3RV/3RV(n = 21) | | | |
| 2R/3R | 0 (0%) | 7 (37%) | 12 (63%) |
| 3R/3R | | | |
| 3R/3RV(n = 19) | | | |
| P = 0.019¹ | | | |

¹Based on Fisher's Exact Test

Comparative analysis of these data indicates that screening for the SNP in combination with the tandem repeat polymorphism is more accurate and effective for predicting clinical outcome to 5-FU based on TS genotype analysis. Including a genotype for the SNP and regrouping patients based on predicted TS expression significantly increased the predictive value of the tandem repeats alone in response to 5-FU/LV chemotherapy (p=0.089 v. 0.019 with SNP), and overall survival (p=0.14 v. 0.035 with SNP).

### Overview of the 3′ UTR Experiment Series

The experiments characterized the 3′UTR of TS, and determined the effects of a −6 bp/1494 deletion polymorphism on TS mRNA stability/and or translational efficiency. Using a luciferase based assay system to determine stability, the experiments showed that the entire 3′UTR of TS was relatively stable overall and contained no elements that caused significant instability or translational repression. It was also determined that the −6 bp/1494 deletion polymorphism was associated with decreased mRNA stability and an enhanced rate of mRNA decay. In addition, the −6 bp/1494 deletion polymorphism is of predictive value in determining the TS mRNA levels of a given individual and that this polymorphism is relatively common, and varies greatly among different ethnic populations. Thus, it is an excellent candidate for use in various cancer screens.

The −6 bp/1494 polymorphism is associated with decreased TS mRNA levels, which leads to TS protein levels being affected similarly. The results are consistent with a recent study involving Japanese patients with rheumatoid arthritis (RA). RA patients that were homozygous for the deletion polymorphism (−6 bp/−6 bp) had a significantly higher incidence of >50% improvement in serum C-reactive protein levels. This indicates response (Nozoe, 1998 and 2001) after treatment with low-dose methotrexate, than individuals bearing any +6 bp alleles (Kumagai, 2003). Another related study screened for the tandem repeat polymorphism along with the newly identified functional G116C SNP that lies within the tandem repeats. That functional SNP was shown to improve the value of the tandem repeats alone in predicting outcome of patients with metastatic colorectal carcinoma treated with a 5-FU based chemotherapy regimen. These findings are consistent with the fact that patients with lower TS expression may be more sensitive to methotrexate, an indirect TS inhibitor, than individuals with higher TS expression.

The functional −6 bp/1494 deletion polymorphism is a candidate for use as a predictor of TS gene expression. Interestingly, a significant linkage disequilibrium was found between the 5′ triple tandem repeat genotype (3R/3R) and the −6 bp/−6 bp 1494 deletion genotype in the Japanese RA study population (Kumagai, 2003). In addition, an earlier study showed the first evidence of linkage disequilibrium between the tandem repeat polymorphisms and −6 bp deletion polymorphisms (Ulrich, 2002).

In this study, a significant linkage disequilibrium was found between 5′ double tandem repeat (2R/2R) genotypes and +6 bp/+6 bp 1494 genotypes in a Caucasian population. These findings are significant due to the influences that these polymorphisms have on TS gene expression and may help explain discrepancies observed when screening individuals for the tandem repeat polymorphism alone. For example, an individual that is homozygous for the 2R tandem repeat polymorphism might display relatively high TS gene expression. This would give the false impression that the 2R polymorphism was associated with high TS expression and possibly increased resistance to 5-FU. Since the 2R tandem repeat polymorphism occurs much more frequently with the +6 bp/1494 polymorphism that stabilizes TS mRNA, screening for both polymorphisms, in conjunction with the recently identified G116C SNP within the tandem repeats resolves many of the discrepancies in correlating genotypes with TS gene expression. Further, the inclusion of the −6 bp/1494 deletion polymorphism in screens using the tandem repeats and G116C SNP may improve the prognostic value of the test in future clinical studies.

1. The 3′UTR of TS is Stable and Contains no Detectable Elements of mRNA Instability or Translational Silencing.

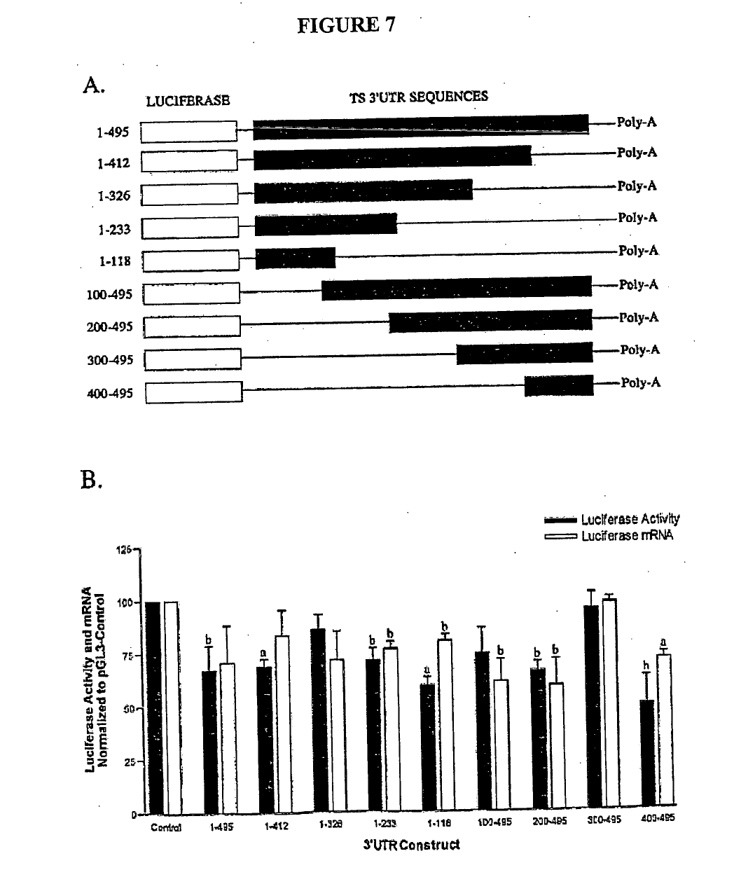

In order to determine the function of the −6 bp/1494 polymorphism, regulatory elements within the entire 3′UTR of TS were characterized. Since the +6 bp/1494 allele is more common in the sample population from which the constructs were amplified, the analysis began with the TS 3′UTR containing the +6 bp/1494 polymorphism. Various reporter constructs were created by inserting regions of the human TS-3′UTR into the 3′UTR of the luciferase gene, in the pGL3-control plasmid (FIG. 7A). By inserting the 3′UTR of TS into the unique XbaI restriction site of the plasmid, each reporter construct was controlled by the SV40 promoter and each contained an SV40 late poly(A) signal downstream of the luciferase 3′UTR. Since each reporter construct differed only by the TS-3′UTR regions that were inserted, changes in luciferase activity should be due to altered post-transcriptional regulation.

293 cells were transiently transfected with each reporter construct, incubated overnight for reporter gene expression, and assayed for luciferase activity. All luciferase values are expressed as a percentage of the luciferase activity from the pGL3-control construct that contained no regions of the TS-3′UTR. Luciferase activity from this construct was designated as 100% activity. Inserting the full length 3′UTR of TS (1-495) into the luciferase 3′UTR resulted in a ˜35% decrease in luciferase activity (FIG. 7B, black bars). The decrease in luciferase activity was expected since the luciferase mRNA is a highly stable transcript on its own, and a similar decrease in luciferase activity compared to control activity has been shown previously in other 3′UTR studies using this system (Cok, 2001; Giles, 2003). Serial deletions from the proximal and distal ends of the TS-3′UTR resulted in similar decreases in luciferase activity overall, and had no additional effect on luciferase activity compared to the full-length (1-495) construct (FIG. 7B, black bars).

In order to determine whether the observed changes in luciferase activity were due to changes in mRNA stability or translational efficiency, luciferase mRNA was quantified. If changes in luciferase activity correlated with changes in luciferase message levels, then alterations seen by insertion of the TS-3′UTR would likely be due to changes in message stability. If luciferase activity did not correlate with luciferase message levels, then changes would likely be due to alterations in translational efficiency.

Cells were lysed after transfection, and total RNA was quantified and used for semi-quantitative RT-PCR. Luciferase mRNA levels were normalized to GAPDH mRNA levels, and were expressed as a percentage of the luciferase message from the pGL3-control constructs bearing no TS-3′UTR sequences. Compared to the empty pGL3-control construct, a significant decrease in luciferase mRNA levels was observed with most TS-3′UTR bearing constructs (FIG. 7B, white bars). However, no significant decreases in message levels were observed between the full-length (1-495) TS-3′UTR construct and each of its deletion constructs. These results correlate with the changes in luciferase activity seen above, and indicate that the decreases in luciferase activity caused by insertion of the TS-3′UTR regions into the highly stable luciferase 3′UTR were mainly due to altered mRNA stability. In addition, since no significant changes in luciferase mRNA levels or luciferase activity were seen between full-length and deletion constructs of the TS-3′UTR, these results indicate that there are no major mRNA instability or translational silencing elements within the TS-3′UTR.

2. TS-3′UTR Constructs Bearing the 6 bp/1494 Deletion Polymorphism have Decreased Luciferase Activity and mRNA Levels Compared to TS-3′UTR Constructs Containing the 6 bp.

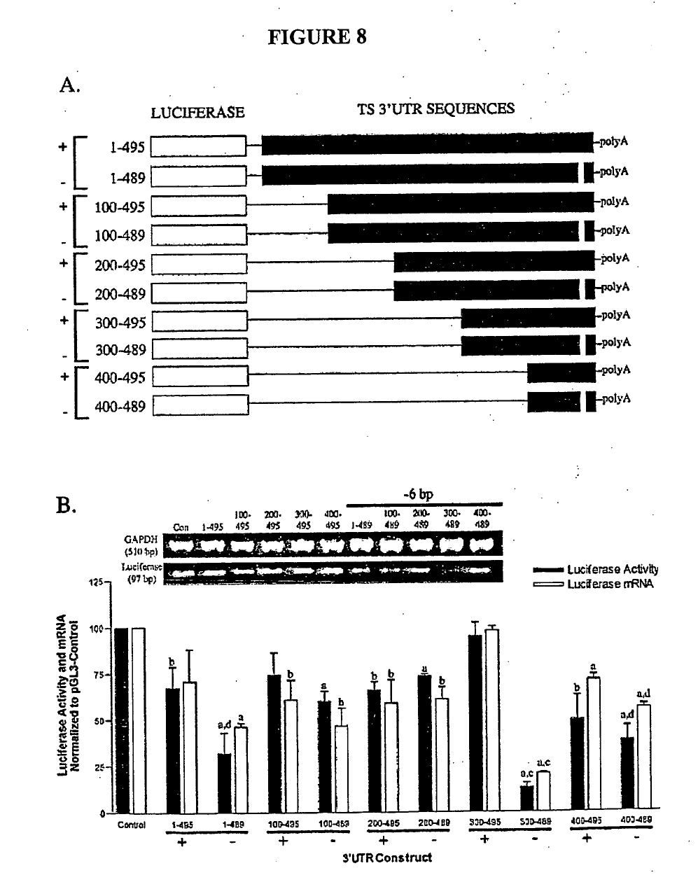

To determine the effects of the −6 bp/1494 deletion on TS-3′UTR regulation, a series of constructs bearing the deletion polymorphism were made. Since the polymorphism lies in the far distal region of the 3′UTR (FIG. 8A, indicated by gaps at nucleotide 456 of 495), it was only necessary to create constructs that were either full-length or serial deletions from the proximal end of the 3′UTR.

293 cells were transiently transfected with the −6 bp/1494 constructs and incubated overnight. Cells were harvested and lysates were assayed for either luciferase activity or luciferase mRNA levels as above. The full-length −6 bp/1494 (1-489) construct had 35% less luciferase activity (p<0.05) compared to its +6 bp/1494 counterpart (1-495) (FIG. 8B, black bars). This decrease in luciferase activity correlated with a similar decrease in mRNA levels (FIG. 8B, white bars), suggesting that the changes in luciferase activity between +6 bp and −6 bp constructs were due to changes in mRNA stability and not translational silencing. Significant differences in luciferase activity and mRNA levels between +6 bp and −6 bp constructs were also observed for the 300-495 vs. 300-489 constructs and the 400-495 vs. 400-489 constructs.

These results further prove that the −6 bp/1494 constructs had significantly decreased mRNA stability, compared with TS-3′UTR constructs bearing the +6 bp. There was no evidence for increased translational repression from the −6 bp/1494 constructs as seen by the correlation between decreases in luciferase activity and mRNA levels.

3. The −6 bp/1494 Deletion in the TS-3′UTR Causes Decreased Message Stability.

In order to support the theory that the decreases in luciferase protein and mRNA levels were due to decreased message stability, an mRNA decay assay was performed. By treating cells with actinomycin D, which inhibits new transcription, one can measure the relative half-life, or rate of mRNA decay, of a given transcript. Cells were transfected with either +6 bp or −6 bp/1494 TS-3′UTR constructs, and were treated with actinomycin D in order to inhibit new transcription. Cells were harvested every 2 hours post-treatment, for 6 hours, and total RNA was obtained and used for RT-PCR analysis of luciferase mRNA levels. Results are displayed as the percentage of mRNA remaining at time zero.

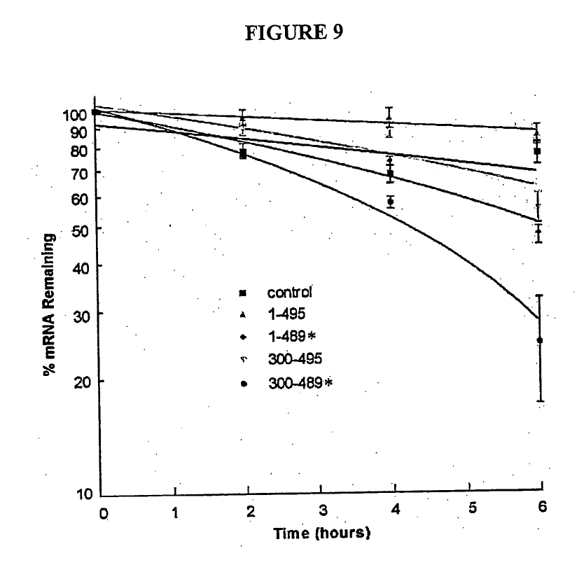

The full-length (1-495) TS-3′UTR construct had 93% mRNA remaining after 6 hours, compared with 70% for pGL3-control (FIG. 9), showing that the TS-3′UTR is highly stable after 6 hours. The −6 bp/1494 (1-489) TS 3′UTR construct had 45% less mRNA remaining after 6 hours compared to its +6 bp/1494 counterpart (p<0.05). The 300-495 (+6 bp) and 300-489 (−6 bp) constructs were also utilized for this assay since they displayed the most robust differences in luciferase activity and mRNA levels. The 300-489 (−6 bp) construct had 38% less mRNA remaining after 6 hours compared with its wild-type 300-495 (+6 bp) counterpart (p<0.05). These results confirm that the decreases in luciferase mRNA levels in TS-3′UTR constructs bearing the −6 bp/1494 deletion polymorphism were due to an increased rate of mRNA degradation and not due to alterations in translational efficiency.

4. The −6 bp/1494 Deletion Polymorphism is Associated with Intratumoral TS mRNA Levels.

Since the in vitro data proved that the −6 bp/1494 polymorphism caused decreased mRNA stability, it was next determined whether this polymorphism was associated with low TS mRNA expression in vivo. Intratumoral TS mRNA expression was measured in 43 individuals with advanced colorectal carcinoma by real-time Taqman RT-PCR and normalized to β actin mRNA. In order to correlate TS gene expression with the 6 bp/1494 polymorphism, these individuals were screened for the polymorphism by RFLP analysis from genomic DNA taken from whole blood as previously described (Ulrich, 2000).

The distribution of genotypes for the polymorphism (Table 3) were 30% homozygous for the insertion (+6 bp/+6 bp), 56% heterozygous for the deletion polymorphism (+6 bp/−6 bp), and 14% homozygous for the deletion polymorphism (−6 bp/−6 bp). The geometric mean of TS mRNA expression (Table 3) was the highest (11.35) in individuals that were homozygous for the insertion (+6 bp/+6 bp), the lowest (2.71) in individuals homozygous for the deletion polymorphism (−6 bp/−6 bp). The value for TS mean expression fell in between the two extremes (5.42) in individuals that were heterozygous for the polymorphism (+6 bp/−6 bp). The comparison of the association between genotype (+6 bp/+6 bp vs. −6 bp/−6 bp) and TS mRNA levels was statistically significant (p=0.007). In addition, the overall comparison between genotypes and TS mRNA levels was statistically significant (p=0.017).

TABLE 3
Measurement of TS mRNA levels in metastatic tumor tissue and
distribution of the −6 bp/1494 deletion polymorphism among 43 Caucasian
individuals with colorectal cancer.

| TS | | | | | | |
|---|---|---|---|---|---|---|
| mRNA | | | TS² | | Comparison of TS Mean | |
| Genotype | n¹ | Genotype (%) | Mean | 95% CI³ | Genotype | p-value⁴ |
| +6 bp/+6 bp | 13 | 30 | 11.35 | (6.43, 20.03) | (+6/+6) vs. (−6/−6) | 0.007 |
| +6 bp/−6 bp | 24 | 56 | 5.42 | (3.57, 8.24) | (+6/+6) vs. (+6/−6) | 0.041 |
| −6 bp/−6 bp | 6 | 14 | 2.71 | (1.18, 6.26) | (+6/−6) vs. (−6/−6) | 0.14 |
| | | | | | Overall | 0.017 |

total number of individuals in sample population.
TS mean = geometric mean of mRNA expression of TS relative to β actin mRNA. 95% confidence interval.
p-value for the overall comparison is based on the F-test, all other p-values are based on the LSD (least significant difference) test.

These findings are consistent with our in vitro data which indicates that the −6 bp/1494 polymorphism is associated with decreased mRNA stability, and provides further in vivo evidence of this association.

5. The −6 bp/1494 Deletion Polymorphism Varies Greatly Among Different Ethnic Populations.

Using the RFLP analysis cited above, the frequency of the −6 bp/1494 deletion polymorphism was assessed in non-Hispanic whites, Hispanic whites, and African-Americans in Los Angeles, Calif. (Table 4).

TABLE 4
Distribution of the 6 bp/1494 deletion polymorphism among
non-Hispanic white, Hispanic white, African-American, and
Singapore Chinese individuals.

| | | | | | Allele | |
|---|---|---|---|---|---|---|
| | | | | | frequency | |
| Ethnic | | Genotype (%) | | | (%) | |
| Group | n¹ | +6 bp/+6 bp | +6 bp/−6 bp | −6 bp/−6 bp | +6 bp | −6 bp |
| White | 63 | 40 | 38 | 22 | 59 | 41 |
| Hispanic | 98 | 58 | 33 | 9 | 74 | 26 |
| Afr. | 59 | 25 | 46 | 29 | 48 | 52 |
| Amer. | | | | | | |
| Chinese | 80 | 50 | 49 | 1 | 74 | 26 |

total number of individuals in sample population

The genotype frequencies of the polymorphism in non-Hispanic whites were 40% homozygous (+6/+6), 38% heterozygous (+6/−6) and 22% homozygous for the deletion polymorphism (−6/−6). The distribution of this polymorphism is consistent with previous reports in Caucasians (Ulrich, 2000; Kumagai, 2003). The genotype frequencies of the polymorphism were 58% (+6/+6), 33% (+6/−6) and 9% (−6/−6) in Hispanic whites, and 25% (+6/+6), 46% (+6/−6) and 29% (−6/−6) in African-Americans. There was a statistically significant difference in genotype frequencies across the three racial-ethnic groups (p<0.0001). When compared by genotype distribution between racial-ethnic groups, two at a time, except for non-Hispanic whites versus African-Americans, all other pair-wise comparisons yielded statistically significant differences.

III. Materials and Methods

Expression, Purification and Phosphorylation of Recombinant USF-1 and Expression of USF-2
cDNA encoding USF-1 (Gregor et al., 1990) was amplified from 34Lu human lung fibroblast cDNA. The upper primer was 5′-CGGGATCCATGAAGGGGCAGCAGAAAACAG-3′ [SEQ ID NO: 2] and lower primer was 5′-GCTCTAGATTAGTTGCTGTCATTCTTGATGACGA-3′ [SEQ ID NO: 3], adds BamHI and XbaI restriction sites respectively. PCR was carried out under the following conditions using Accuzyme DNA Polymerase (Bioline): 30 cycles for 30 s at 94° C., 30 s at 59.3° C., and 45 s at 72° C. The product was digested with BamHI and XbaI and cloned in-frame into the pProEX-HTb vector (Invitrogen) that adds a 6-histidine tag to the N-terminus of the expressed protein. The plasmid was transformed into the DH5α strain of E. coli (Invitrogen) and protein expression was induced by adding IPTG to a final concentration of 0.6 mM to the culture. After induction, the cells were centrifuged at 10,000×g for 10 min and resuspended in 4 volumes of lysis buffer (20 mM Tris-HCl, pH 8.5 at 4° C., 100 mM KCl, 5 mM 2-mercaptoethanol, 1 mM PMSF). Cells were lysed in a French press and cell debris was removed by centrifugation. Supernatant was run on a Ni-NTA resin column following the pProEX-HT Prokaryotic Expression System protocol (Invitrogen) to isolate recombinant 6-histidine tagged USF-I.

To activate the DNA binding ability of USF-1, the recombinant protein was phosphorylated in vitro using cdc2/p34. Cdc2/p34 was isolated by immunoprecipitation using mouse monoclonal antibodies (sc-54, Santa Craz Biotechnologies). An in vitro phosphorylation reaction was carried out by adding 6 μl of 5X-cdc2 kinase buffer (1 M Tris-HCl, pH 7.5, 1 M MgCl₂, and 1 M dithiothreitol), 1 μl of 1 mM ATP/1 mM MgCl₂ (1 mM[γ-³²P]ATP/1 mM MgCl₂ for visualization of phosphorylation), 200 ng of recombinant USF-1, and 8 μl H₂₀ to 15 μl of the protein A sepharose beads bound with cdc2/p34. The reaction was carried out for 20 min at 30° C., then loaded and run on a 12.5% SDS-PAGE gel. The gel was dried and placed in a cartridge with Kodak Biomax Maximum Sensitivity film for visualization of [γ-³²P] ATP incorporation.

USF-2 cDNA was amplified from 34Lu cDNA (upper primer 5′-CCGGAATTCCATGCCATGGACATGCTGGACCC-3′ [SEQ ID NO: 4] and lower primer5′-GCTCTAGACATGTGTCCCTCTCTGTGCTAAGG-3′ [SEQ ID NO: 5], adds EcoRI and XbaI restriction sites respectively) and PCR was carried out under the following conditions using Accuzyme DNA Polymerase (Bioline, Denville Scientific): 30 cycles for 30 s at 94° C., 30 s at 62° C., and 45 s at 72° C. The USF-1 and USF-2 cDNAs were cloned into the pCI-neo plasmid vector (Promega) for expression in transient transfection experiments.

Electrophoretic Mobility Shift Assay (EMSA)

Synthetic double-stranded oligonucleotides (Integrated DNA Technologies) corresponding to a wild type (R) or variant (RV) 28 bp tandem repeat sequence from the TS 5′ regulatory region were labeled with [γ-³²P] ATP (Amersham Pharmacia Biotech) according to the Gel-Shift Assay Kit protocol (Promega). For each gel shift reaction, 10,000 cpm of labeled probe 30 ng of recombinant USF-1 for 20 min at room temperature in a 20 μl reaction mixture containing 10 mM Tris-HCl, pH 7.5, 50 mM NaCl, 0.5 mM dithiothreitol, 0.5 mM EDTA, 4% glycerol, 1 mM MgCl₂ and 0.1 μg of poly(dIdC) DNA. Where indicated, unlabeled competitor oligonucleotides were incubated for 10 min at room temperature with nuclear extracts prior to addition of labeled probe.

Reactions using recombinant USF-1 contained ˜30 ng of either USF-1 or phospho USF-1. Samples were loaded onto a non-denaturing 4% acrylamide gel and electrophoresed in 0.5×TBE buffer at 350 V at 4° C. The gels were dried and visualized by autoradiography using Kodak BioMax Maximum Resolution Film. Sequences of the oligonucleotides were as follows: TS wild-type repeat (R), 5′-CCGCGCCACTTGGCCTGCCTCCGTCCCG-3′ [SEQ ID NO: 6]; TS variant repeat (RV), 5′-CCGCGCCACTTcGCCTGCCTCCGTCCCG-3′ [SEQ ID NO: 7]; USF-1 specific competitor oligonucleotide (Santa Cruz Biotechnology), 5′-CACCCGGTCACGTGGCCTACACC-3′ [SEQ ID NO: 8]; USF-1 mutant competitor oligonucleotide (Santa Cruz Biotechnology), 5′-CACCCGGTCAATTGGCCTACACC-3′ [SEQ ID NO: 9]; poly (dIdC) (Sigma) used as non-specific competitor.

Chromatin Immunoprecipitation Assay (ChIP)

Chromatin immunoprecipitation from 293 cells was carried out using the ChIP assay kit (Upstate Biotechnologies) according to the manufacturer's protocol. Briefly, 1×10⁶ cells were plated in 10 cm dishes and incubated overnight at 37° C. The cross-linking of protein to DNA was carried out by adding 37% formaldehyde to the growth medium at a final concentration of 1%. The cross-linking reaction was performed for 10 min at 37° C. Cells were washed in ice-cold PBS containing protease inhibitors (Protease inhibitor cocktail set III, Calbiochem) and scraped into conical screw-cap tubes. Cells were centrifuged and resuspended in SDS lysis buffer, then sonicated 3 times for 10 seconds at full power on ice, using a Branson 450 sonifier, to shear DNA to 200-1,000 bp fragments. Samples were centrifuged and 200 μl of sonicated cell supernatant was diluted into 1,800 μl of ChIP dilution buffer for each protein of interest.

Salmon sperm DNA bound to Protein A agarose was added and spun down to remove non-specific background. The rabbit polyconal immunoprecipitating antibodies (USF-1, sc-229×; USF-2, sc-861×, Santa Cruz Biotechnologies) were added to each tube and incubated overnight at 4° C. with rotation. Salmon sperm DNA/Protein A agarose was added for 1 hr at 4° C. and pelleted to isolate the antibody/protein/histone/DNA complexes. The protein-DNA complexes were washed and eluted, and the cross-linking was reversed by heating samples at 65° C. for 4 hours. DNA was recovered by phenol/chloroform extraction and ethanol precipitation. PCR was carried out using the same primers and conditions as in the RFLP protocol.

Construction of Reporter Plasmids

The TS promoter, located in the genomic sequence upstream of the 5′-exon of the gene, was identified and isolated. Primers were designed at −313 and +195 relative to transcription start and the PCR reaction yielded a 508 bp product for the 3R genotype and a 480 bp product for the 2R genotype. In order to isolate 3RV DNA, PCR amplification was performed from a random population of human genomic DNA and products were sequenced directly (Davis Sequencing). Fragments were cloned into the promoter-less pGL3-Basic luciferase reporter gene vector (Promega) at SstI and XhoI sites just upstream of luciferase gene transcription start. Site-directed mutagenesis was carried out according to the manufacturer's protocol (Promega) to alter the USF-1 E-box consensus elements within the first 28 bp tandem repeat of both the 2R and 3R constructs. The mutagenic oligonucleotide primer sequence was 5′-GTCCTGCCACCGCGCgtCTTGGCCTGCC-3′ [SEQ ID NO: 10] (Integrated DNA Technologies) and yielded the 2RmutUSF and 3RmutUSF reporter constructs. All plasmid DNA was isolated and purified using Qiagen mini- and Midi-prep kits.

Cell Culture and Transient Transfections

Human embryonic kidney 293 cells (American Type Culture Collection) were plated in 6-well dishes at a density of 5×10⁵ cells/well and incubated overnight in 2.5 ml of DMEM medium supplemented with 5% (v/v) fetal bovine serum, 100 units/ml penicillin, 100 μg/ml streptomycin, 10 mM pyruvate and 2 mM L-glutamine. The next day, growth media was aspirated from the cells and replaced with 2.5 ml of serum-free Opti-MEM medium (Invitrogen). A total of 5 μg of plasmid DNA (1 μg of pCMV-β-galactosidase (Invitrogen) for standardization of transfection efficiencies, 1 μg of USF-1/pCI-neo or pCI-neo, and 3 μg of reporter construct) was diluted into 250 μl of Opti-MEM. A solution containing 250 μl of Opti-MEM and 15 μl of Lipofectamine 2000 reagent (Invitrogen) was incubated for 5 min at room temperature and mixed with the DNA containing solution from the previous step. After a twenty-minute incubation at room temperature, the DNA-Lipofectamine solution was added drop-wise to the 293 cells in a circular fashion and cells were incubated for 2 hr at 37° C. The solution was aspirated and replaced with 3 ml of growth medium and cells were incubated overnight at 37° C. to allow gene expression.

Cell Transfection

293 cells were transfected with 3 μg of luciferase reporter construct containing either no promoter (pGL3-Basic), the TS 5′ region containing two tandem repeats (2R), the TS 5′ region containing three tandem repeats (3R), the TS 5′ region containing the 3R with a G→C SNP at the 12^{th} nucleotide of the third repeat, or the TS 5′ region containing two or three tandem repeats with mutated E-box sites (2RmutUSF and 3RmutUSF). Cells were co-transfected with 1 μg of empty pCI-NEO vector, 1 μg of vector containing USF-1 cDNA, 1 μg of vector containing USF-2 cDNA, or 0.5 μg of USF-1 and USF-2 containing vectors, respectively. Cells were also co-transfected with 1 μg of the pCMV-β-galactosidase vector for standardization of transfection efficiencies. Twenty-four hours after transfection, cells were harvested, lysed, and assayed for β-galactosidase activity and luciferase activity. Results from these experiments show that there was an increase in relative luciferase activity from both the 2R and 3R constructs in the presence of USF-1.

Luciferase Assay

Luciferase activity was determined using a luciferase assay system (Promega) following the manufacturer's protocol. Briefly, cells were scraped into lysis reagent, transferred to microfuge tubes and centrifuged for 30 s at 12,000×g. Luciferase activity was measured using a manual luminometer (Turner Design, TD 20/20) by mixing 100 μl of luciferase assay reagent with 20 μl of 1:10 diluted cell lysate and reading three times at 10 sec intervals for each sample. Transfection efficiencies were obtained using a P-galactosidase assay (Promega) of cell lysates by reading the absorbance at 420 nm. Relative luciferase activity was quantified by standardizing luciferase activity to a transfection efficiency factor.

Genotyping of 2R, 3R and 3RV by Restriction Fragment Length Polymorphism Analysis

Genomic DNA was isolated from 100 colorectal cancer patients from 200 μl of whole blood using the QiaAmp kit (Qiagen, Valencia, Calif.). To isolate the region of DNA containing the tandem repeats, PCR primers were designed at +15 and +195 relative to transcription start. The upper primer sequence was 5′-CGAGCAGGAAGAGGCGGAG-3′ [SEQ ID NO: 1] and the lower primer sequence was 5′-TCCGAGCCGGCCACAGGCAT-3′ [SEQ ID NO: 12]. 35 cycles of PCR were carried out for 30 s at 94° C., 30 s at 60° C., and 1 m at 72° C. 15 μl of the PCR reaction was digested with HaeIII restriction enzyme in a 20 μl reaction volume. The digested and undigested PCR products from each patient were loaded into adjacent lanes on a 3% sea plaque agarose (BioWhittaker Molecular Applications) gel containing ethidium bromide (0.5 mg/ml) and electrophoresed in 0.5×TBE. Genotyping was performed twice for all samples by independent investigators.

Patient Selection

The presence of the new SNP within the TS gene was confirmed in Non-Hispanic Whites. Individuals (disease-free controls) were initially recruited for a cancer case-control study in California as described in Castelao et al., 2001. Patients included in this study has metastatic colorectal cancer and were enrolled in the following protocols: Southwest Oncology Group protocol 9420 (19 patients, 5-FU doses used: CI (continuous infusion) 300 mg/m²/d v. CI 2600 mg/m²/d q weekly) opened for accrual in May 1995 and closed in May 1999; and the University of Southern California protocol: 3C-92-2 (21 patients, 5-FU doses used: CI 200 mg/m²/d q weekly for three weeks followed by one week rest) opened for accrual in September 1992 and closed in June 1995. All patients signed an informed consent to participate in the clinical trial and for evaluation of the TS polymorphism. Genotyping for the TS polymorphism was performed on paraffin-embedded tissues in all patients.

All patients had bi-dimensionally measurable disease at the time of protocol entry. Responders to therapy were classified as those patients whose tumor burden (the sum, overall measurable lesions of the products of the largest diameter and its perpendicular diameter) decreased by 50% or more for at least six weeks. Progressive disease was defined as 25% or more increase in tumor burden (compared to the smallest measurement) or the appearance of new lesions. Patients that did not experience a response and did not progress within the first 12 weeks following the start of 5-FU/luecovorin were classified as having stable disease.

Survival was computed as the number of months from the initiation of chemotherapy with 5-FU to death of any cause. Patients who were alive at the last follow-up evaluation were censored at that time.

Statistical Analysis

Contingency tables and Fisher's exact test (Metha et al., 1983) were used to summarize the association of response (grouped as response, stable disease, and progressive disease) to 5-FU with the TS genotypes. Kaplan-Meier plots (Kaplan et al., 1958) and the log-rank test (Miller et al., 1981) were used to compare survival of patients according to TS genotypes. Median survival was calculated based on the Kaplan-Meier estimator. All p-values are two-sided.

Preparation and Study of Six Base Pair Deletion in the 3′ UTR Cell Culture

The 293 human embryonic kidney (HEK) cell line was obtained from ATCC and cells were cultured in Dulbecco's modified Eagle medium supplemented with 5% (v/v) fetal bovine serum, 100 units/ml penicillin, 100 μg/ml streptomycin, 10 mM pyruvate and 2 mM L-glutamine. Cells for all experiments were confluent, and used within 10 passages of the original stock supplied by ATCC.

Reporter Gene Construction

Various regions of the thymidylate synthase gene encoding the 3′UTR were amplified from genomic DNA by PCR using primers terminating in an SpeI recognition sequence which produces compatible ends with XbaI digested fragments. Products containing the +6 bp/1494 polymorphism and the −6 bp/1494 polymorphism were obtained through PCR by pre-screening human genomic DNA for homozygous template samples for each polymorphism by RFLP analysis as previously described (Ulrich, 2000). DNA fragments were digested, purified by agarose gel electrophoresis and extracted using a DNA gel extraction kit (Millipore). PCR products were ligated into the pGL3-control vector (Promega) within the 3′-UTR of the firefly luciferase gene at a unique XbaI site. The orientation, sequence, and 1494 polymorphisms of all constructs were confirmed by sequencing (Davis Sequencing).

Transient Transfections

293 cells were transiently transfected using the LipofectAMINE 2000 transfection reagent (Invitrogen). Cells were plated in six-well plates at a density of 1×10⁶ cells/well and incubated overnight. Transfections were carried out following the manufacturer's protocol (Invitrogen). 1.5 μg of reporter gene plasmid DNA, and 0.5 μg of pCMV-β-galactosidase plasmid DNA (Invitrogen) for standardization, were mixed in 500 μl of serum-free medium with 4 μl of transfection reagent and incubated for 20 minutes at room temperature. The DNA-LipofectAMINE complex was added dropwise to each well and cells were incubated overnight for gene expression. Cells were either treated with actinomycin D, or lysed directly in culture dishes for luciferase assays or mRNA quantitation, 24 hours post-transfection.

Luciferase Assays

Luciferase activity was determined using a luciferase assay system (Promega) following the manufacturer's protocol. 350 μl of cell culture lysis reagent was added to each well and cells were scraped and transferred to microfuge tubes. Cellular debris was removed by centrifugation for 2 minutes at 12,000 rpm. Supernatant was diluted 1:10 in cell culture lysis reagent and assayed for luciferase activity using a manual luminometer (TD 20/20, Turner Designs).

Luciferase assays were performed by mixing 100 μl of luciferase assay reagent with 20 μl of diluted supernatant. Light output was measured over a ten-second time period in triplicate for each sample. Relative luciferase activity was calculated by averaging the readings and then normalizing to transfection efficiencies by measuring β-Galactosidase activity. Relative β-galactosidase activity was measured using an assay kit (Promega) and by determining the absorbance of samples at 420 nm. All luciferase values are expressed as the percentage of relative luciferase activity compared to pGL3-control.

Semi-Quantitative Reverse Transcriptase-PCR

Total RNA was isolated from transfected cells using the RNeasy Mini Kit (Qiagen). Total RNA was treated with DNase I while on the mini-columns to eliminate amplification of reporter plasmid DNA and genomic DNA. Total RNA was quantified and normalized for amplification by RT-PCR using the One Step RT-PCR Kit (Qiagen). cDNA was run on a 2% agarose gel and band intensity of luciferase and glyceraldehyde-3-phosphate (GAPDH) products was quantified by densitometry using Eagle Eye software (Stratagene). Luciferase amplification primers were 5′-GCCTGAAGTCTCTGATTAAGT-3′ [SEQ ID NO: 13] for the forward primer and 5′-ACACCTGCGTCGAAGATGT-3′ [SEQ ID NO: 14] for the reverse primer (97 bp product).

Amplification primers for GAPDH were CCCCTGGCCAAGGTCATCCATGACAACTTT [SEQ ID NO: 15] for the forward primer and GGCCATGAGGTCCACCACCCTGTTGCTGTA [SEQ ID NO: 16] for the reverse primer (510 bp product). 15 pmol of each luciferase primer and 3 pmol of each GAPDH primer (internal control) were used in each reaction. The PCR conditions consisted of: Hot start at 50° C. for 30 min for the RT reaction and 95° C. for 15 min followed by 25 cycles of 1 min at 94° C., 1 min at 58° C., 1:30 min at 72° C., followed by 72° C. for 10 min. The amount of luciferase message in each RNA sample was quantified and normalized to GAPDH content and is expressed as a percentage of luciferase cDNA compared to cells transfected with the pGL3-Control vector.

Reporter Gene mRNA Decay

293 (HEK) cells were transiently transfected and incubated for 24 hours to allow for luciferase gene expression. Media was aspirated and replaced with media containing actinomycin D (10 μg/ml) in order to inhibit new transcription. Total RNA was isolated at various times points after actinomycin D treatment, and luciferase mRNA content was determined by RT-PCR as described above. Luciferase mRNA levels were normalized to GAPDH mRNA content and are expressed as a percentage of the mRNA level present at time zero. Data were plotted by linear regression analysis using the Prism program (Graph Pad, Inc.).

Statistical Analysis

All experiments were performed on three separate occasions, each in duplicate. Data are expressed as the means±S.E. Comparison of means was performed using the Student's t test.

Patient Selection, mRNA Quantitation, and Statistical Analysis

The 43 patients in this study had advanced colorectal carcinoma and were previously untreated. All patients signed an informed consent for tissue collection and evaluation of determinants of 5-FU efficacy and toxicity. A PCR amplification and RFLP analysis was performed to identify the TS 6 bp/1494 genotypes of each patient as previously described (Ulrich, 2000). TS mRNA was measured using a quantitative RT-PCR method as described in detail elsewhere (Horikoshi, 1992).

Allelic Frequency Analysis

TS genotype measurements were performed on 63 non-Hispanic white, 98 Hispanic white, and 59 African-American subjects in Los Angeles, Calif. and on 80 Chinese subjects in Singapore using an RFLP based analysis as previously described (Ulrich, 2000). The 63 non-Hispanic white subjects represented a random sample of the 691 white controls from a recently completed population-based case-control study of bladder cancer in Los Angeles County (Castelao, 2001). The 59 African-American (34 bladder cancer cases plus 25 controls) and 98 Hispanic (50 bladder cancer cases plus 48 controls) subjects also were participants of this Los Angeles Bladder Cancer Study (Castelao, 2001). Among African-American or Hispanic white subjects, there was no statistically significant difference in genotypic distributions between bladder cancer cases and controls. Therefore, frequencies were reported for all subjects combined within each race. The 80 Singapore Chinese subjects were a random sample of the 63,000 participants of the Singapore Chinese Health Study, an ongoing prospective cohort study focusing on diet and cancer development (Seow, 2002). The chi-square test was used to examine possible differences in genotype distributions by race. All p-values quoted are two-sided. p-values less than 0.05 are considered statistically significant.

1. An isolated thymidylate synthase nucleic acid molecule of SEQ ID NO: 1, wherein G is replaced by C at nucleotide 12.

2. (canceled)

3. A single-stranded nucleic acid probe that hybridizes to the isolated nucleic acid molecule of claim 1, but not to SEQ ID NO: 1.

4-5. (canceled)

6. A diagnostic kit comprising the probe of claim 3, or an allele-specific nucleic acid primer of 8-40 nucleotides that specifically hybridizes to and detects a thymidylate synthase nucleic acid molecule of SEQ ID NO: 1, wherein G is replaced by C at nucleotide 12, and instructions for use.

7-10. (canceled)

11. A method for determining whether an individual has or has a heightened predisposition to cancer or cardiovascular disease, comprising:
(a) obtaining a sample from the individual comprising a thymidylate synthase nucleic acid molecule; and
(b) detecting one or more polymorphisms in the thymidylate synthase nucleic acid molecule, wherein
(i) an individual with an 3R/3R construct in the 5′ region of the thymidylate synthase nucleic acid molecule has or has a heightened predisposition to cancer or cardiovascular disease as compared to an individual with a 3R/3RV, 2R/2R, 2R/3R, or 2R/3RV construct;
(ii) an individual with a +6 bp/1494 3′ untranslated region polymorphism of the thymidylate synthase nucleic acid molecule has a heightened predisposition to cancer or cardiovascular disease as compared to an individual with a −6 bp/1494 3′ untranslated region polymorphism of the thymidylate synthase nucleic acid molecule;
(iii) an individual with both the 3R/3R construct in the 5′ region and a +6 bp/1494 3′ untranslated region polymorphism of the thymidylate synthase nucleic acid molecule has or has the highest probability of developing cancer or cardiovascular disease.

12-20. (canceled)
